(19) United States
(12) Patent Application Publication
(10) Pub. No.: US 2025/0303126 A1
(43) Pub. Date: Oct. 2, 2025

(54) CARDIOVASCULAR IMPLANT DEVICES FOR DIRECTING FLOW

(71) Applicant: EDWARDS LIFESCIENCES CORPORATION, Irvine, CA (US)

(72) Inventors: Sakyasingh Tripathy, San Diego, CA (US); Douglas Thomas Dominick, Irvine, CA (US); Hengchu Cao, Irvine, CA (US); Alexis Rodriguez, San Antonio, TX (US); Koohyar Vahidkhah, Rancho Santa Margarita, CA (US); Koray Senol, Laguna Niguel, CA (US); Yousef Naranjani, Lake Forest, CA (US); Bingquan Su, Irvine, CA (US)

(73) Assignee: EDWARDS LIFESCIENCES CORPORATION, Irvine, CA (US)

(21) Appl. No.: 19/231,280

(22) Filed: Jun. 6, 2025

Related U.S. Application Data
(63) Continuation of application No. PCT/US2023/084313, filed on Dec. 15, 2023.
(60) Provisional application No. 63/387,917, filed on Dec. 16, 2022.

(51) Int. Cl. A61M 27/00

(52) U.S. Cl. CPC A61M 27/002; A61M 2206/20; A61M 2210/125

(57) ABSTRACT

A cardiovascular implant device includes an annular body, one or more anchoring members, and a flow directing component. The annular body includes a central flow tube extending from an inflow end to an outflow end and a flow path extending through the central flow tube. The one or more anchoring members extending outward from the annular body and are configured to secure the cardiovascular implant device to a tissue wall. The flow directing component is positioned to align a flow of blood out of the cardiovascular implant device with a natural flow pattern of blood in a right atrium so that the flow of blood out of the cardiovascular implant device joins with the natural flow pattern of blood in the right atrium.

### CROSS-REFERENCE TO RELATED APPLICATION(S)

This application is a continuation of International Application No. PCT/US2023/084313, filed Dec. 15, 2023, which claims the benefit of U.S. Provisional Application No. 63/387,917, filed Dec. 16, 2022, the disclosures of which are hereby incorporated by reference in their entireties.

### BACKGROUND

The present disclosure relates to cardiovascular implant devices, and more specifically to cardiovascular implant devices for directing flow.

Cardiovascular implant devices can be positioned in natural flow paths within the cardiovascular system or can be used to create artificial flow paths. For example, shunt devices can be positioned the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt devices can be positioned in the inter-atrial septal wall between the left atrium and the right atrium to shunt blood from the left atrium into the right atrium, thus reducing the pressure in the left atrium.

### SUMMARY

In one example, a cardiovascular implant device includes an annular body, one or more anchoring members, and a flow directing component. The annular body includes a central flow tube extending from an inflow end to an outflow end and a flow path extending through the central flow tube. The one or more anchoring members extending outward from the annular body and are configured to secure the cardiovascular implant device to a tissue wall. The flow directing component is positioned to align a flow of blood out of the cardiovascular implant device with a natural flow pattern of blood in a right atrium so that the flow of blood out of the cardiovascular implant device joins with the natural flow pattern of blood in the right atrium.

In another example, a cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The central flow tube includes a curved portion adjacent the outflow end, the curved portion being curved to align a flow of blood out of the cardiovascular implant device with a natural flow pattern of blood in a right atrium so that the flow of blood out of the cardiovascular implant device joins with the natural flow pattern of blood in the right atrium.

In another example, a cardiovascular implant device includes an annular body, one or more anchoring members, and a flap. The annular body includes a central flow tube extending from an inflow end to an outflow end and a flow path extending through the central flow tube. The one or more anchoring members extend outward from the annular body and are configured to secure the cardiovascular implant device to a tissue wall. The flap is connected to the annular body at the outflow end of the central flow tube. The flap is angled to align a flow of blood out of the cardiovascular implant device with a natural flow pattern of blood in a right atrium so that the flow of blood out of the cardiovascular implant device joins with the natural flow pattern of blood in the right atrium.

In another example, a cardiovascular implant device is configured to be attached adjacent to an opening in a tissue wall between a right atrium and a left atrium of a heart. The cardiovascular implant device includes an anchoring member configured to secure the cardiovascular implant device to the tissue wall, a flexible joint connected to the anchoring member, and a flap connected to the flexible joint. The flap is angled to align a flow of blood out of the opening with a natural flow pattern of blood in the right atrium so that the flow of blood out of the puncture joins with the natural flow pattern of blood in the right atrium.

In another example, a cardiovascular implant device includes an annular body and one or more anchoring members. The annular body includes a central flow tube extending from an inflow end to an outflow end, which includes a guide wall connected to a radially inner surface of the central flow tube, and a flow path extending through the central flow tube and defined by the guide wall. The one or more anchoring members extend outward from the annular body and are configured to secure the cardiovascular implant device to a tissue wall. The guide wall is positioned to guide a flow of blood through the central flow tube of the cardiovascular implant device so that a flow of blood out of the cardiovascular implant device aligns and joins with a natural flow pattern of blood in a right atrium.

In another example, a cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The cardiovascular implant device further includes a shaft extending longitudinally through the central flow tube and a set of blades extending radially about the shaft. The blades are positioned to guide a flow of blood through the central flow tube of the cardiovascular implant device so that a flow of blood out of the cardiovascular implant device aligns and joins with a natural flow pattern of blood in a right atrium.

In another example, a cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The central flow tube includes an adjustable portion adjacent the outflow end. The adjustable portion is adjustable between one or more expanded configurations and a compressed configuration and is adjustable to align a flow of blood out of the cardiovascular implant device with a natural flow pattern of blood in a right atrium so that the flow of blood out of the cardiovascular implant device joins with the natural flow pattern of blood in the right atrium.

In another example, a cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The central flow tube is configured to be angled with respect to the tissue wall. The central flow tube is angled to align a flow of blood out of the cardiovascular implant device with a natural flow pattern of blood in a right atrium so that the flow of blood out of the cardiovascular implant device joins with the natural flow pattern of blood in the right atrium.

### BRIEF DESCRIPTION OF THE DRAWINGS

### Anatomy of Heart H and Vasculature V

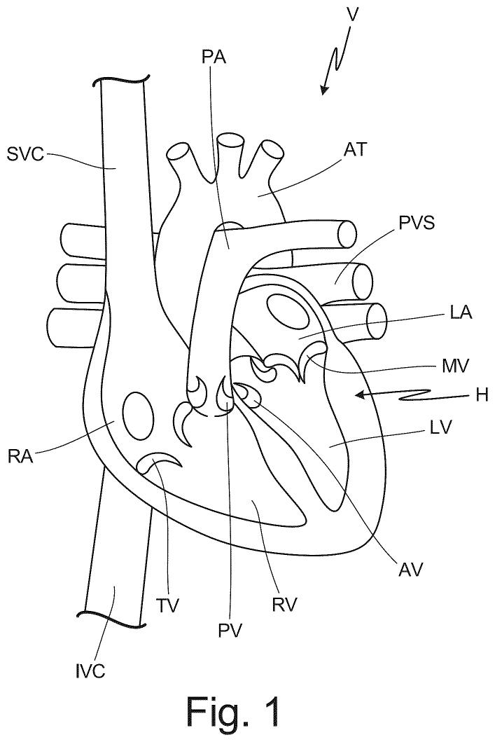

FIG. 1 is a schematic diagram of a heart and vasculature.

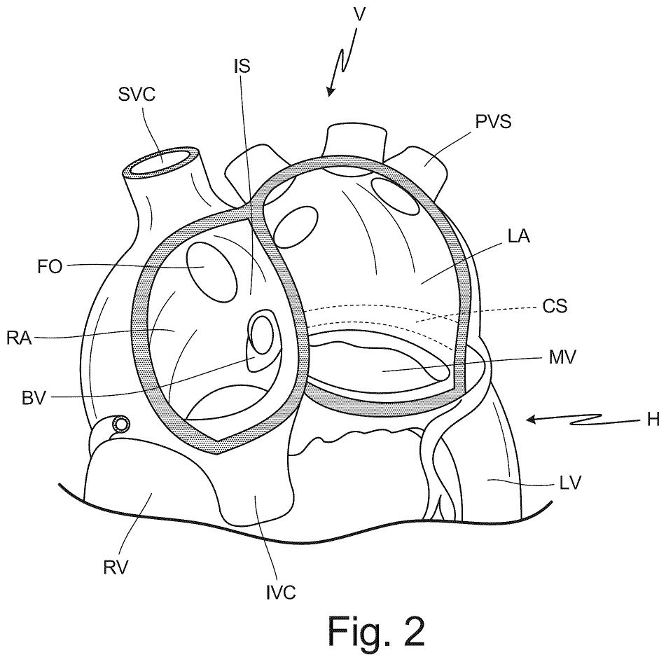

FIG. 2 is a schematic cross-sectional view of the heart.

### Flow Patterns of Heart H

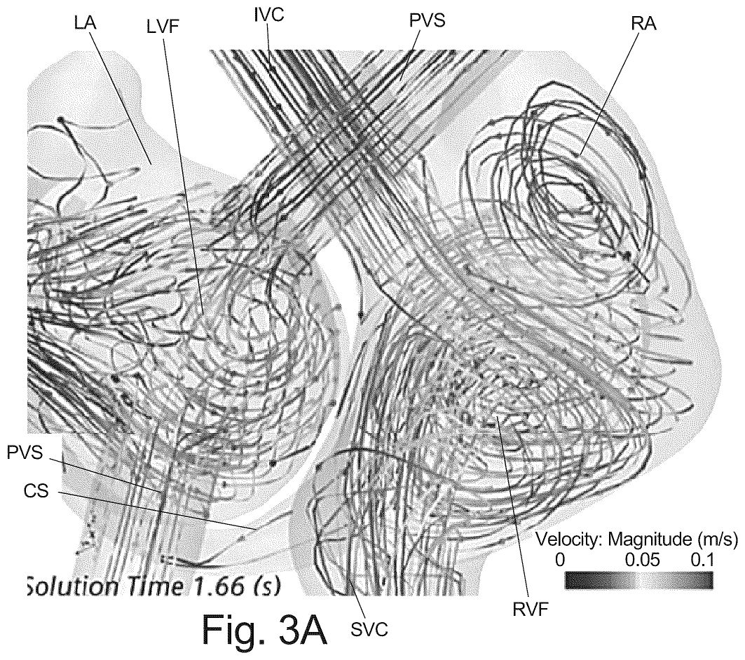

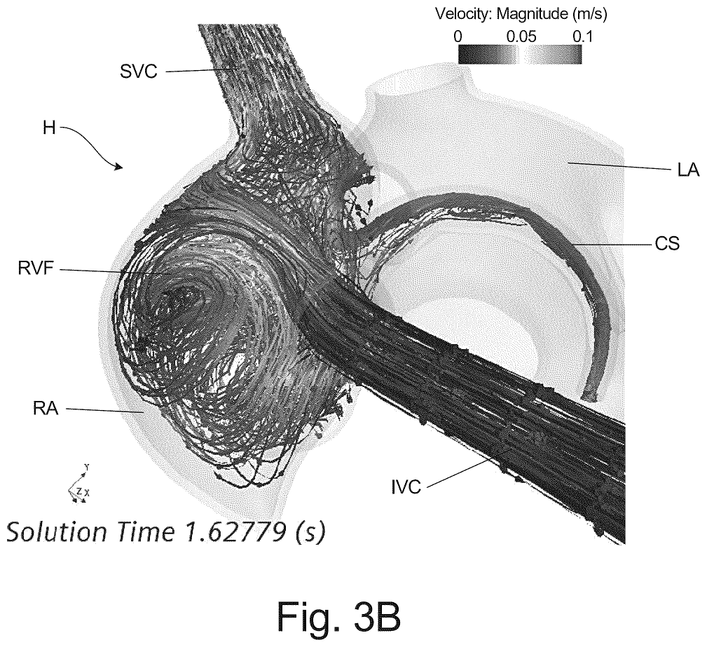

FIG. 3A is a first schematic diagram illustrating modeled hemodynamic flow patterns in a heart.

FIG. 3B is a second schematic diagram illustrating modeled hemodynamic flow patterns in a heart.

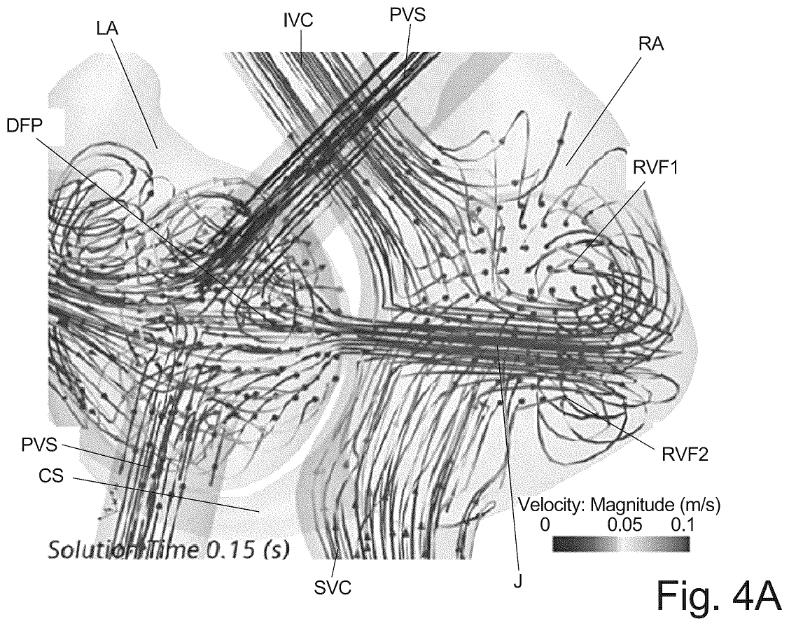

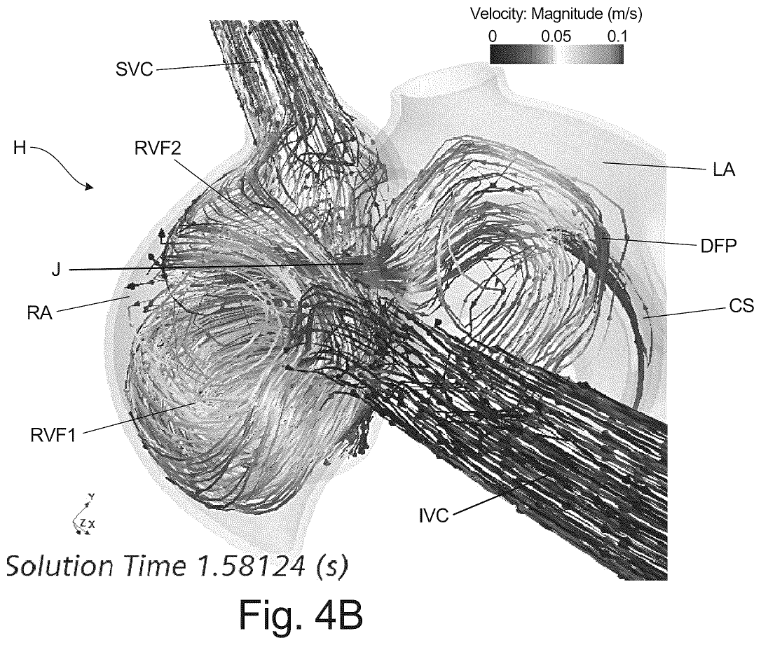

FIG. 4A is a first schematic diagram illustrating modeled hemodynamic flow patterns in a heart with a septal shunt device.

FIG. 4B is a second schematic diagram illustrating modeled hemodynamic flow patterns in a heart with a septal shunt device.

### Devices 100 and 100A

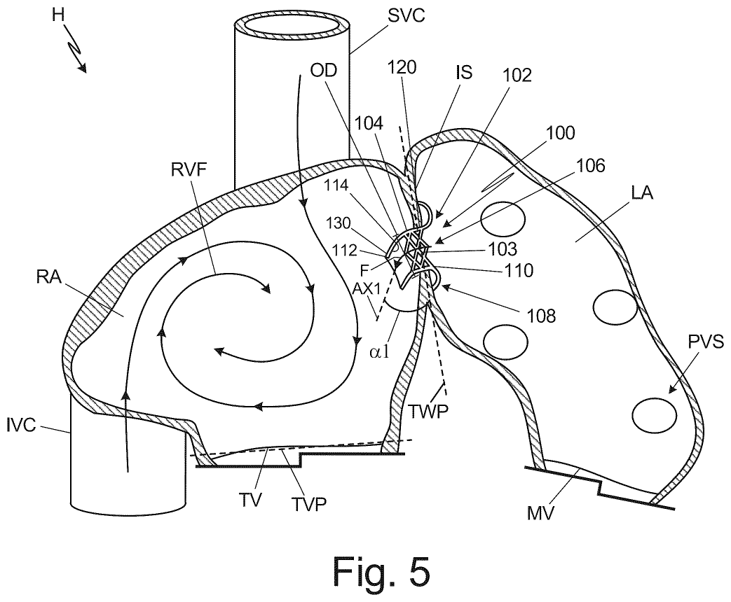

FIG. 5 is a schematic cross-sectional view of a heart illustrating a first example of a cardiovascular implant device positioned in an inter-atrial septum and including a curved portion.

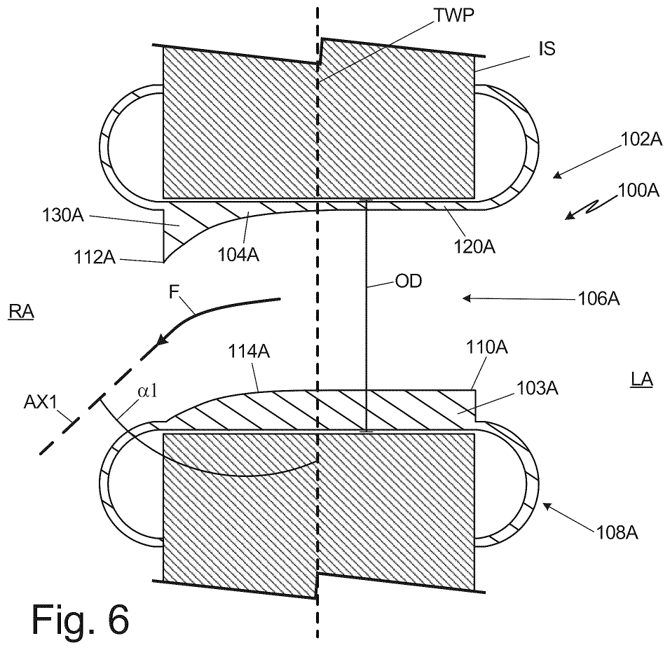

FIG. 6 is a schematic cross-sectional view of an inter-atrial septum illustrating a second example of a cardiovascular implant device positioned in the inter-atrial septum and including an internal curved portion.

### Device 200

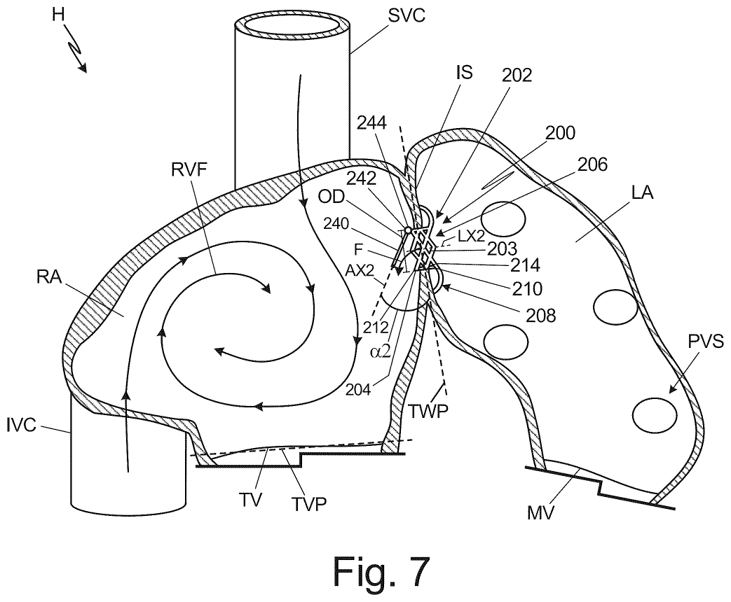

FIG. 7 is a schematic cross-sectional view of a heart illustrating a third example of a cardiovascular implant device positioned in an inter-atrial septum and including a flap.

### Device 300

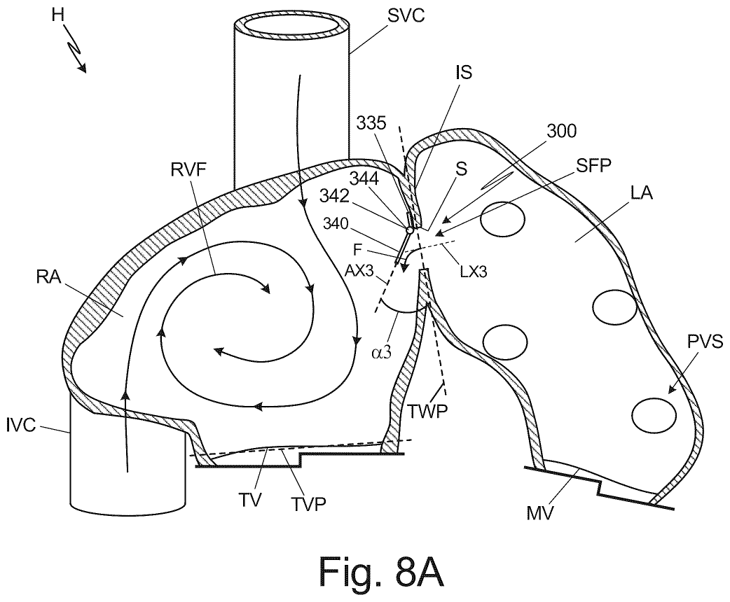

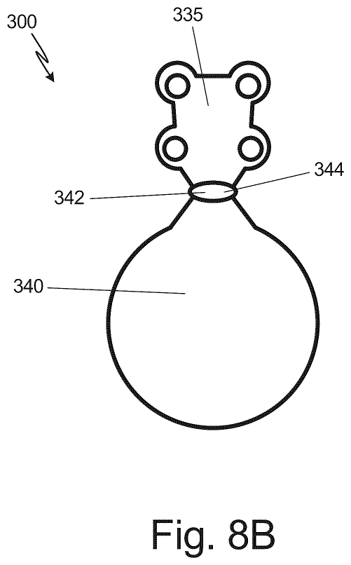

FIG. 8A is a schematic cross-sectional view of a heart illustrating a fourth example of a cardiovascular implant device positioned adjacent a shunt in an inter-atrial septum.

FIG. 8B is an enlarged side view of the fourth example of the cardiovascular implant device.

### Device 400

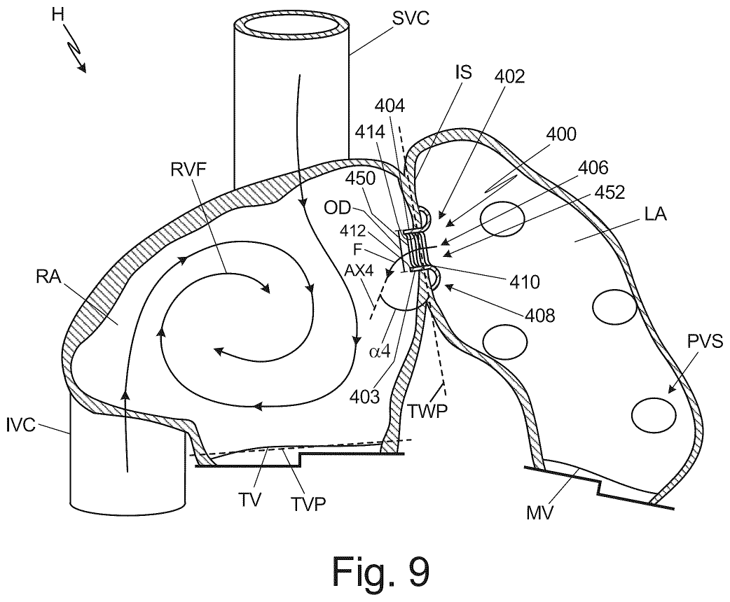

FIG. 9 is a schematic cross-sectional view of a heart illustrating a fifth example of a cardiovascular implant device positioned in an inter-atrial septum including an internal guide wall.

### Device 500

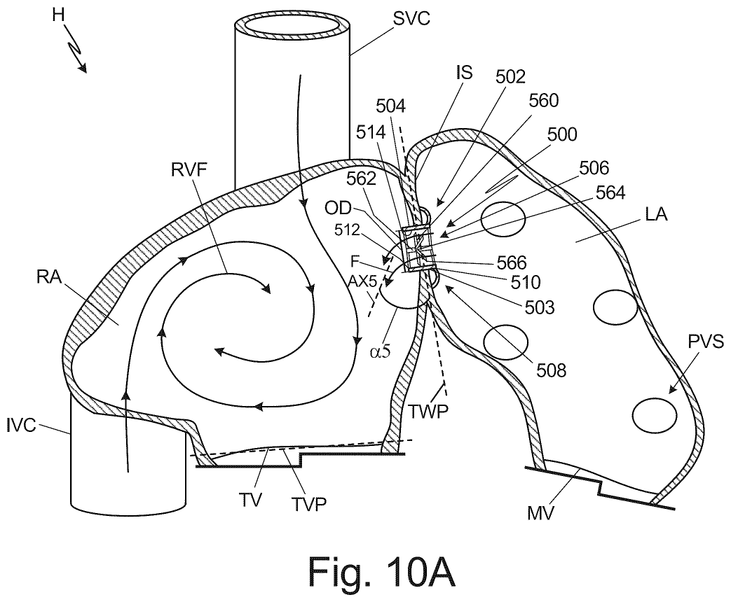

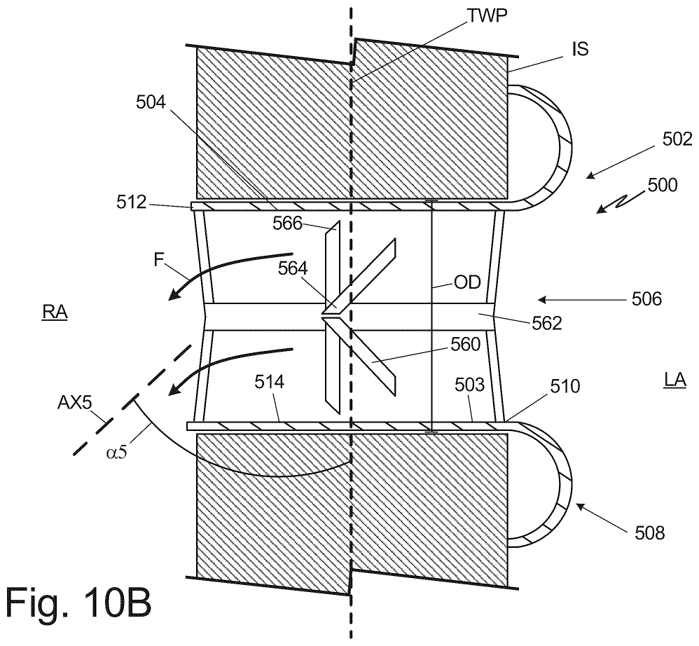

FIG. 10A is a schematic cross-sectional view of a heart illustrating a sixth example of a cardiovascular implant device positioned in an inter-atrial septum and including internal blades.

FIG. 10B is an enlarged schematic cross-sectional view of an inter-atrial septum illustrating details of the sixth example of the cardiovascular implant device.

### Devices 600 and 600A

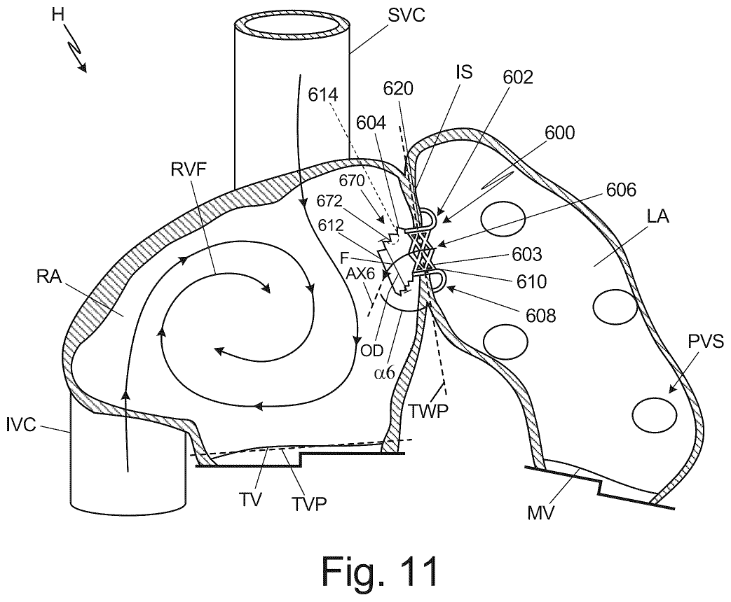

FIG. 11 is a schematic cross-sectional view of a heart illustrating a seventh example of a cardiovascular implant device positioned in an inter-atrial septum and including an adjustable portion.

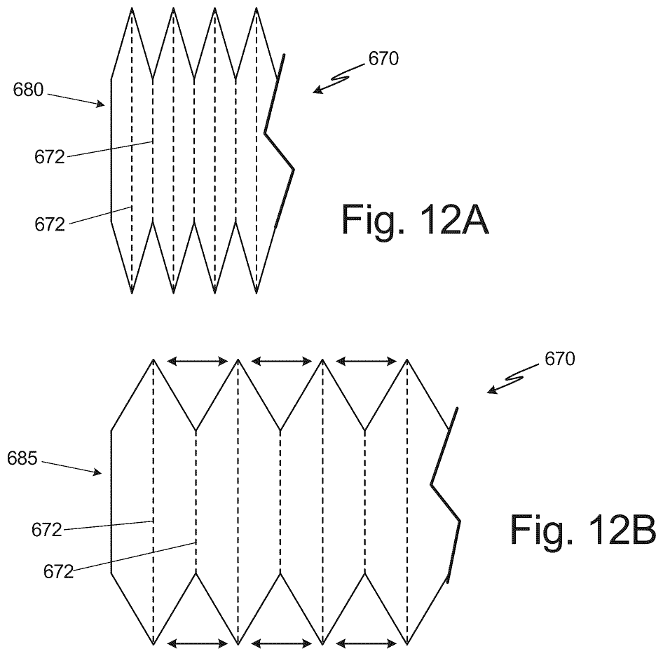

FIG. 12A is an enlarged schematic view of the adjustable portion of FIG. 10 in a compressed configuration.

FIG. 12B is an enlarged schematic view of the adjustable portion of FIG. 10 in an expanded configuration.

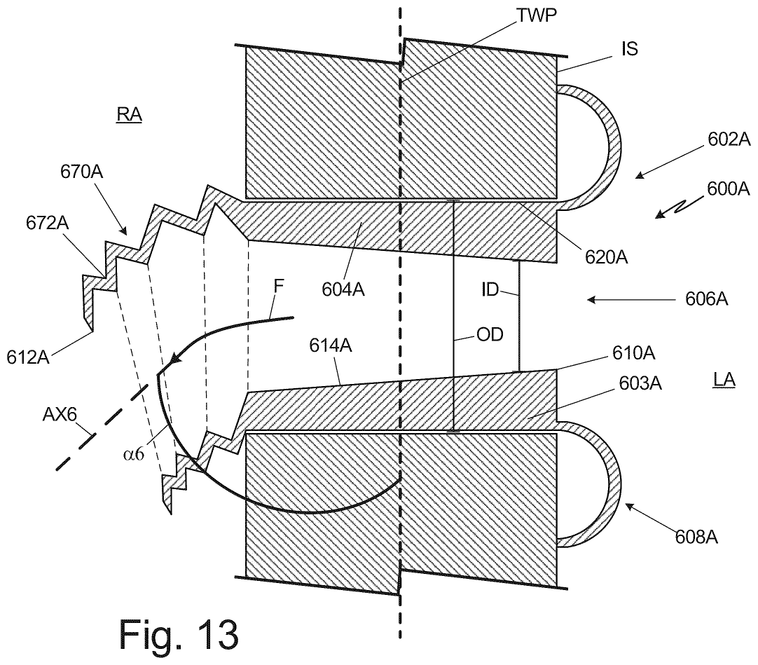

FIG. 13 is a schematic cross-sectional view of an inter-atrial septum illustrating an eighth example of a cardiovascular implant device positioned in the inter-atrial septum and including a varying inner diameter.

### Device 700

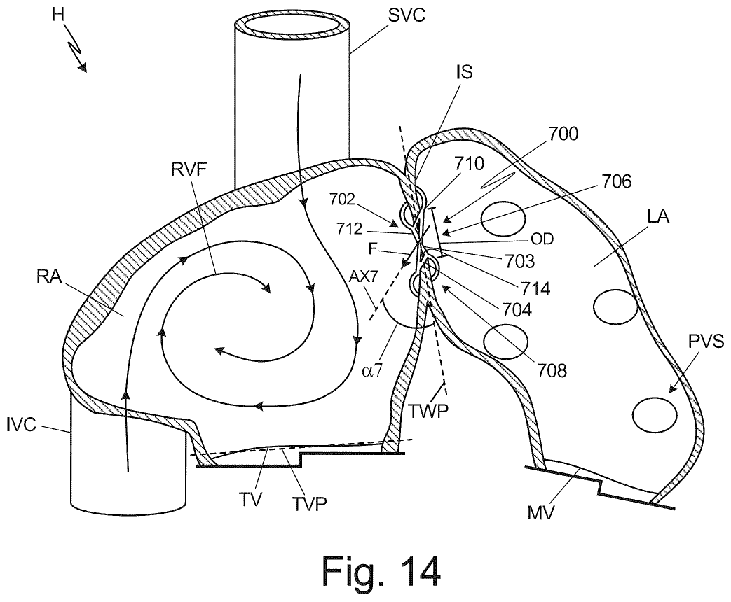

FIG. 14 is a schematic cross-sectional view of a heart illustrating a ninth example of a cardiovascular implant device positioned in an inter-atrial septum and including an angled central flow tube.

### DETAILED DESCRIPTION

### Anatomy of Heart H and Vasculature V (FIGS. 1-2)

FIG. 1 is a schematic diagram of heart H and vasculature V. FIG. 2 is a cross-sectional schematic view of heart H. FIGS. 1-2 will be discussed together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC, inferior vena cava IVC, and coronary sinus CS.

A majority of the blood flows into right atrium RA from superior vena cava SVC and inferior vena cava IVC, which are offset from one another. Due to the offset of the major entry blood flows from superior vena cava SVC and inferior vena cava IVC, a natural flow vortex occurs in right atrium RA (a right-sided flow vortex). This allows a substantial portion of blood from right atrium RA to pass through right atrium RA and enter right ventricle RV by direct flow. The right-sided flow vortex in right atrium RA preserves kinetic energy and momentum of the major blood flows entering right atrium RA and allows a substantial portion of blood to naturally pass from right atrium RA to right ventricle RV without any contribution to flow needed from the pumping action of right atrium RA. With contraction, right atrium RA also pumps the residual portion of the entering blood not caught in the direct flow through tricuspid valve TV into right ventricle RV. The blood enters right ventricle RV and then flows through pulmonary valve PV into pulmonary artery PA. With preservation of direct inflow from right atrium RA, blood entering right ventricle RV also forms a natural flow vortex (a right-ventricular flow vortex) in right ventricle RV, which naturally re-directs blood entering right ventricle RV to pulmonary artery PA by direct flow without requiring right ventricle RV to perform substantial work of pumping blood. Residual blood that is not transported to pulmonary artery PA via pulmonary valve PV by direct flow is pumped by the contraction of right ventricle RV. The blood flows from pulmonary artery PA into smaller arteries that deliver the deoxygenated blood to the lungs via the pulmonary circulatory system. The lungs can then oxygenate the blood.

The left side of heart H, including left atrium LA and left ventricle LV, receives the oxygenated blood from the lungs and provides blood flow to the body. Blood flows into left atrium LA from pulmonary veins PVS. The offset of the right and left pulmonary veins PVS also leads to the formation of a natural flow vortex in left atrium LA (left-sided flow vortex), which helps maintain momentum and minimize work as the blood traverses left atrium LA to mitral valve MV. Direct flow, as described above, and the pumping action of left atrium LA propels the blood through mitral valve MV into left ventricle LV. As the blood enters left ventricle LV, a natural flow vortex (a left-ventricular flow vortex) forms in left ventricle LV, which redirects flow naturally towards the left ventricular outflow of aortic valve AV so that it can be efficiently pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it approaches right atrium RA. Coronary sinus CS also wraps around a portion of right atrium RA posteriorly before in enters right atrium RA via the ostium of coronary sinus CS lateral and posterior to an orifice of tricuspid valve TV, and medial to inferior vena cava IVC entry point. Due to its proximity to inferior vena cava IVC, blood entering right atrium RA from coronary sinus CS is naturally entrained into the larger inflow from inferior vena cava IVC forming the natural flow vortex (right-sided flow vortex) in right atrium RA, which naturally redirects the inflows towards tricuspid valve TV.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood, primarily from inferior vena cava IVC and coronary sinus CS, to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and enters the inferior vena cava IVC via a natural shunt called the ductus venosus. The oxygenated blood moves through inferior vena cava IVC to right atrium RA. The opening of inferior vena cava IVC in right atrium RA is positioned to direct the oxygenated blood through right atrium RA and then through a second natural shunt called foramen ovale into left atrium LA along with the entrained deoxygenated blood from coronary sinus CS. Left atrium LA can then pump the mixed oxygenated and deoxygenated blood into left ventricle LV, which pumps it to aorta AT and the systemic circulatory system. This allows the pulmonary circulatory system to be bypassed in utero. Some deoxygenated blood, primarily from superior vena cava SVC, is pumped through the right heart where it also bypasses the lungs and reenters aorta AT via a third natural shunt called the ductus arteriosus. Upon birth, respiration expands the lungs, blood begins to circulate through the lungs to be oxygenated, and the three natural shunts close. The closure of the foramen ovale forms fossa ovalis FS.

Shunt devices can be positioned in heart H to shunt blood between left atrium LA and right atrium RA. Left atrium LA has a higher pressure and lower compliance compared to right atrium RA, and right atrium RA has a lower pressure and higher compliance than left atrium LA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema. Reduction of back pressure on the pulmonary circulation also reduces pulmonary artery PA pressures, which can injure the small arteries leading to the lungs resulting in pulmonary hypertension. Increased pulmonary artery pressures can also lead to pressure overload of right ventricle RV, injuring right ventricle RV and potentially leading to right sided heart failure.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of left ventricle LV and certain forms of congenital heart and valve disease.

Septal shunt devices (also called inter-atrial shunt devices or trans-septal shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow primarily from right atrium RA to left atrium LA. Shunt devices can also be left atrium to coronary sinus shunt devices that are positioned in a tissue wall between left atrium LA and coronary sinus CS where the two structures are in close approximation as coronary sinus CS passes through the atrio-ventricular groove that is covered by epicardium. Left atrium to coronary sinus shunt devices move blood from left atrium LA into coronary sinus CS, which then delivers the blood to right atrium RA via the ostium of coronary sinus CS, the natural orifice of coronary sinus CS, which may have thebesian valve BV. Coronary sinus CS is compliant and can quickly grow in response to increased volume with conditions such as drainage of the left subclavian vein to coronary sinus CS. Similarly, coronary sinus CS can act as an additional compliance chamber when using a left atrium to coronary sinus shunt device. In general, shunt devices can potentially affect the natural flow patterns in vessels and/or chambers of heart H. These flow patterns will be discussed below in greater detail with respect to FIGS. 3A-4B.

### Flow Patterns of Heart H (FIGS. 3A-4B)

FIG. 3A is a first schematic diagram illustrating modeled hemodynamic flow patterns in heart H. FIG. 3B is a second schematic diagram illustrating modeled hemodynamic flow patterns in heart H. FIG. 4A is a first schematic diagram illustrating modeled hemodynamic flow patterns in heart H with a septal shunt device. FIG. 4B is a second schematic diagram illustrating modeled hemodynamic flow patterns in heart H with a septal shunt device. FIGS. 3A-4B show heart H, right atrium RA, left atrium LA, superior vena cava SVC, inferior vena cava IVC and coronary sinus CS. FIGS. 3A and 4A also show tricuspid valve TV, pulmonary veins PVS, and mitral valve MV.

FIGS. 3A and 4A represent a computational fluid dynamics model of right and left atria. The anatomical geometry was generated from segmenting and averaging computerized tomography (CT) data from pre-implant patients. Shunt geometry (included in FIG. 4A) was virtually added to the model. A computational mesh consisting of polyhedral cells was created on the geometry, and boundary conditions were applied in the form of flow waves at the inlets (pulmonary veins PVS, inferior vena cava IVC, superior vena cava SVC, and coronary sinus CS) and pressure waves at the outlets (mitral valve MV and tricuspid valve TV planes). Blood was modeled as a Newtonian viscous fluid with a density of 1050 kg/m3 and a viscosity of 0.0035 Pascal-second (Pa·s). A K-epsilon (k-ε) Reynolds Averaged Navier-Stokes (RANS) turbulence model was employed in a segregated flow solver in which time and space were discretized in first and second order accuracy respectively. Multiple cardiac cycles were modeled to remove any initial transience and achieve fully periodic flow characteristics. Flow was visualized by generating streamlines at different time instances during a cardiac cycle using the post-processing tools available in the CFD software. FIGS. 3A and 4A show velocity streamlines at a particular time instant during the cardiac cycle.

FIGS. 3A-4B show modeled velocity stream lines representing hemodynamic flow patterns in heart H. FIGS. 3A and 4A show heart H oriented with right atrium RA on a right side of the figures and left atrium LA on a left side of the figures. FIGS. 3A and 4A are inferior views of heart H. FIGS. 3B and 4B show heart H oriented with right atrium RA on a left side of the figures and left atrium LA on a right side of the figures. FIGS. 3B and 4B are superior views of heart H.

Natural flow patterns of blood flow exist in heart H and help move blood through heart H and into the vasculature connected to heart H in a way that maximizes preservation of blood flow momentum and kinetic energy. The natural flow pattern for blood moving through arteries and veins is typically helical in nature (helical flow patterns). The natural flow pattern for blood moving through the chambers of heart H is typically vortical in nature (vortical flow patterns).

FIG. 3A shows modeled hemodynamic flow patterns that exist in right atrium RA and left atrium LA of heart H. FIG. 3B shows modeled hemodynamic flow patterns that exist in right atrium RA, superior vena cava SVC, inferior vena cava IVC, and coronary sinus CS. FIGS. 3A-3B represent natural flow patterns that are formed in heart H, including right atrium RA and left atrium LA, based on the offset of inflows of blood into the chambers of heart H in addition to the anatomical structure of heart H. When looking at heart H from the right side (the right sagittal view), a clockwise right-sided flow vortex is formed in right atrium RA and a counter-clockwise left-sided flow vortex is formed in left atrium LA. The right-sided flow vortex in right atrium RA is the natural flow pattern of blood flow in right atrium RA. The left-sided flow vortex in left atrium LA is the natural flow pattern of blood flow in left atrium LA. The modeled hemodynamic flow patterns shown in FIGS. 3A-3B represent intra-cardiac flow patterns for a structurally normal heart.

Blood flows enter the right atrium RA from superior vena cava SVC, inferior vena cava IVC, and coronary sinus CS. The superior vena cava opening and the inferior vena cava opening in right atrium RA are offset so that the blood flowing into right atrium RA from superior vena cava SVC and inferior vena cava IVC do not collide with each other. Due to its orientation and physical proximity, coronary sinus CS flow is entrained into inferior vena cava IVC flow. The blood flowing through superior vena cava SVC and inferior vena cava IVC has a helical flow pattern. A majority of the blood in right atrium RA enters right atrium RA through inferior vena cava IVC, and the blood flowing from inferior vena cava IVC into right atrium RA is pointed towards the top of right atrium RA. The helical flow pattern of the blood flowing into right atrium RA from inferior vena cava IVC helps to form a clockwise right-sided flow vortex in right atrium RA (when looking at the heart from the right side). The flow of blood entering right atrium RA from superior vena cava SVC will flow along the inter-atrial septum and towards tricuspid valve TV. The helical flow pattern of the blood flowing from superior vena cava SVC into right atrium RA helps the flow of blood naturally join with the clockwise right-sided flow vortex formed in right atrium RA by the flow of blood from inferior vena cava IVC, which is joined by coronary sinus CS flow. A small amount of blood flows into right atrium RA from coronary sinus CS. The flow flowing through coronary sinus CS will have a helical flow pattern. The helical flow pattern of the blood exiting coronary sinus CS will naturally join with inferior vena cava IVC flow and the right-sided flow vortex in right atrium RA. The right-sided flow vortex in right atrium RA is shown with velocity stream lines labeled RVF in FIGS. 3A-3B.

The right-sided flow vortex formed in right atrium RA helps the blood flow through right atrium RA, through tricuspid valve TV, into the right ventricle, through the pulmonary valve, and into the pulmonary artery. The right heart is an inefficient pump and can act more like a conduit. The right-sided flow vortex formed in the right heart helps to preserve kinetic energy and the momentum of blood flow as it moves from superior vena cava SVC and inferior vena cava IVC (the Vena Cavae) through the right heart and into the pulmonary artery, even with minimal to no pumping being provided by the right heart. This is especially important for maintaining right heart output, which must match left heart output, during periods of high output and heart rates during exercise. The right-sided flow vortex formed in right atrium RA helps to move the blood from right atrium RA through tricuspid valve TV and into the right ventricle with minimal loss of momentum and kinetic energy. The blood shoots from right atrium RA through the right ventricle, out the right ventricular outflow tract, through the pulmonary valve, and into the pulmonary artery. Approximately 50% of the blood will flow into the pulmonary artery without any pumping required by the right heart because of the right-sided flow vortices of right atrium RA and right ventricle RV and anatomical constraints of the right heart. Right heart contraction enhances the flow of residual blood through the right heart.

Blood flows into left atrium LA from pulmonary veins PVS. There are four pulmonary veins PVS that flow into left atrium LA. The blood flowing through pulmonary veins PVS has a helical flow pattern. The offset of helical flow of the blood flowing from pulmonary veins PVS into left atrium LA helps to form a counter-clockwise left-sided flow vortex (when looking at the heart from the right side) in left atrium LA. The left-sided flow vortex in left atrium LA directs flow towards mitral valve MV. The left-sided flow vortex in left atrium LA is shown with velocity stream lines labeled LVF in FIG. 3A.

It is hypothesized that if the intra-cardiac blood flow patterns in heart H (including right-sided flow vortex in right atrium RA and left-sided flow vortex in left atrium LA) are disrupted, blood flow from superior vena cava SVC and inferior vena cava IVC (the Vena Cavae), through right atrium RA, through the right ventricle, and into the pulmonary artery, and blood flow from the pulmonary veins, through the left atrium LA, through the left ventricle, and into the aorta become less efficient and place increased mechanical workloads on the respective ventricles. This is especially important in already failing hearts, where the ability to increase the workload of the heart muscle is impaired. Disruptions in the intra-cardiac blood flow patterns in heart H (including right-sided flow vortex in right atrium RA and left-sided flow vortex in left atrium LA) can happen for a variety of reasons. For example, the anatomy of heart H can change as patients age. This can affect the offset between the opening of superior vena cava SVC and the opening inferior vena cava IVC. The blood flow entering right atrium RA from superior vena cava SVC and the blood flow entering right atrium RA from inferior vena cava IVC can collide as the anatomy of heart H changes, which disrupts the natural formation of the right-sided flow vortex in right atrium RA. In another example, right atrium RA can be enlarged in patients with heart failure with or without atrial fibrillation. The enlargement of right atrium RA can also disrupt the right-sided flow vortex formed in right atrium RA. Similarly, left atrium LA can be enlarged in patients with heart failure with or without atrial fibrillation. The enlargement of left atrium LA can disrupt the left-sided flow vortex formed in left atrium LA. Additionally, patients with a patent foramen ovale (a natural inter-atrial septal shunt) or a secundum atrial septal defect due to failure of the patent foramen ovale to fully close may not have the expected intra-cardiac blood flow patterns (including right-sided flow vortex in right atrium RA and left-sided flow vortex in left atrium LA), including the expected flow vortexes created during atrial filling. Closure of a secundum atrial septal defect with altered right atrial non-single vortex flow patterns has been shown to revert to a dominant single vortical flow pattern after the atrial septal defect is occluded.

When the right-sided flow vortex in right atrium RA changes, momentum and energy of the blood flow are lost, and the right heart needs to pump harder to move the blood from right atrium RA into the right ventricle and the pulmonary artery. This is due to the right-sided flow vortex contributing less to the movement of blood through the right heart. Similarly, when the left-sided flow vortex in left atrium LA changes, the left heart needs to pump harder to move the blood from left atrium LA into the left ventricle and the aorta. This is due to the left-sided flow vortex contributing less to the movement of blood through the left heart. Further, as the intra-cardiac flow patterns in heart H (including right-sided flow vortex in right atrium RA and left-sided flow vortex in left atrium LA) change due to age or disease, areas of turbulence can be created in the flow patterns of heart H and there can be a loss of fluid dynamics leading to inefficiencies that could lead to diminished flow. This can increase the susceptibility of the right heart and/or the left heart to fail (the inability to pump enough blood to meet the body's oxygen demands), as heart H has to do more work to move the same amount of blood through heart H. More work is needed to recreate the lost momentum naturally preserved by the intra-cardiac flow patterns in heart H (including right-sided flow vortex in right atrium RA and left-sided flow vortex in left atrium LA), putting additional strain on heart H.

Changes in intra-cardiac flow patterns change intra-cardiac energetics. Heart H is uniquely designed to maximize efficiency by preserving the kinetic energy and momentum of blood flow, thus minimizing the work needed to propagate the blood flow into the chambers, between the chambers, and out of the chambers. Anything that disrupts the intra-cardiac flow patterns in heart H (including right-sided flow vortex in right atrium RA and left-sided flow vortex in left atrium LA) can reduce the efficiency of the energetics of heart H due to a loss of potential energy, which makes it more difficult for heart H to do its job of propagating blood into, between, and out of the chambers. Anything that disrupts the intra-cardiac flow patterns through heart H (including right-sided flow vortex in right atrium RA and left-sided flow vortex in left atrium LA) can increase the amount of work heart H has to do, prolong transit times through heart H, and makes it more difficult for heart H to eject blood. This is especially problematic for people experiencing heart failure, as the heart failure can be exacerbated due to disruptions in the intra-cardiac flow patterns through heart H (including right-sided flow vortex in right atrium RA and left-sided flow vortex in left atrium LA).

FIG. 4A shows modeled hemodynamic flow patterns that exist in right atrium RA and left atrium LA of heart H when a traditional septal shunt device (e.g., a septal shunt device without any additional flow directing or conditioning features) is positioned between right atrium RA and left atrium LA. FIG. 4B shows modeled hemodynamic flow patterns that exist in right atrium RA, superior vena cava SVC, inferior vena cava IVC, coronary sinus CS, and left atrium LA when a traditional septal shunt device is positioned between right atrium RA and left atrium LA. A traditional septal shunt device has been modeled in the inter-atrial septum between right atrium RA and left atrium LA in the schematic shown in FIGS. 4A-4B to allow blood to shunt directly from left atrium LA to right atrium RA.

As shown in FIGS. 4A-4B, when a traditional septal shunt device is positioned in the inter-atrial septum between right atrium RA and left atrium LA, blood jets from left atrium LA into and across right atrium RA. The jet of blood is shown with velocity stream lines labeled J in FIGS. 4A-4B. The jet of blood in right atrium RA disrupts the right-sided flow vortex in right atrium RA. When the blood jets across right atrium RA, two separate flow vortices are formed in right atrium RA. The first flow vortex is shown with velocity stream lines labeled RVF1 and the second flow vortex in shown with velocity stream lines labeled RVF2 in FIGS. 4A-4B. There is also a disruption of the left-sided flow vortex in left atrium LA. The traditional septal shunt device is not aligned with the left-sided flow vortex in left atrium LA, but the pressure difference between right atrium RA and left atrium LA causes the blood in left atrium LA to move out of the left-sided flow vortex and through the septal shunt device into right atrium RA. The disrupted left-sided flow vortex in left atrium LA is shown with velocity stream lines labeled DFP in FIGS. 4A-4B. This disruption of the right-sided flow vortex in right atrium RA and the left-sided flow vortex in left atrium LA can also lead to loss of right ventricle RV and left ventricle LV vortex formations and will cause heart H to have to work harder to pump blood through their respective ventricles and can lead to the development or worsening of heart failure over time.

Specifically, when looking at the right heart, a traditional septal shunt device introduces a significant disruption to the right-sided flow vortex in right atrium RA as the blood jets across right atrium RA. It is hypothesized that the disruption to the right-sided flow vortex in right atrium RA can cause or exacerbate right heart failure. Disruption of the right-sided flow vortex in right atrium RA means that the momentum and kinetic energy of blood naturally or efficiently flowing from right atrium RA into the right ventricle and the pulmonary artery is lost. In order to move the blood from right atrium RA into the right ventricle and the pulmonary artery, the right heart has to work harder to pump the blood. This increased work required by the right heart can cause or exacerbate right heart failure and places a severe load on the less efficient right heart during periods of exercise, where heart rates are high and diastolic filling periods are short.

Several examples of features of cardiovascular implant devices (including several shunt devices) according to techniques of this disclosure will be described with reference to FIGS. 5-14. Each cardiovascular implant device example shown in FIGS. 5-14 includes several generally similar components, which share the same name and which are identified by shared reference numbers that are increased incrementally between each of FIGS. 5-14 (e.g., FIGS. 5-6 include cardiovascular implant devices 100 and 100A; FIG. 7 includes cardiovascular implant device 200;

FIGS. 8A-8B include cardiovascular implant device 300; FIG. 9 includes cardiovascular implant device 400; FIGS. 10A-10B include cardiovascular implant device 500; FIGS. 11-13 include cardiovascular implant devices 600 and 600A; and FIG. 14 includes cardiovascular implant device 700). For ease of discussion, details of some components of the cardiovascular implant device examples shown in FIGS. 5-14 may not be repeated in each of the following sections, but it should be understood that the cardiovascular implant device examples shown in FIGS. 5-14 can include all or any combination of the components and features described herein. Additionally, although depicted in FIGS. 5-14 as separate examples, a cardiovascular implant device according to techniques of this disclosure can generally include any combination of the following features.

The cardiovascular implant devices described herein—cardiovascular implant devices 100, 100A, 200, 300, 400, 500, 600, 600A, 700—can be formed in a variety of ways, e.g., connecting individual wires together to form a mesh or lattice, braiding, cutting from a sheet and then rolling or otherwise forming into the shape of the cardiovascular implant device, molding, cutting from a cylindrical tube (e.g., cutting from a nitinol tube), other ways, or a combination of these. All or a portion of cardiovascular implant devices 100, 100A, 200, 300, 400, 500, 600, 600A, 700 can be made from a flexible metal, metal alloy, polymer, or other suitable material. Examples of metals and metal alloys that can be used include, but are not limited to, nitinol (a nickel titanium alloy) and other shape-memory materials, elgiloy, and stainless steel, but other metals and resilient or compliant non-metal materials can be used to make cardiovascular implant devices 100, 100A, 200, 300, 400, 500, 600, 600A, 700 or their constituent components. All or a portion of cardiovascular implant devices 100, 100A, 200, 300, 400, 500, 600, 600A, 700 can be monolithically formed of any of these materials. These materials can allow cardiovascular implant devices 100, 100A, 200, 300, 400, 500, 600, 600A, 700 to be compressed to a small size, and then—when the compression force is released—cardiovascular implant devices 100, 100A, 200, 300, 400, 500, 600, 600A, 700 can self-expand back to the pre-compressed shape. Cardiovascular implant devices 100, 100A, 200, 300, 400, 500, 600, 600A, 700 can expand back to the pre-compressed shape due to the material properties of cardiovascular implant devices 100, 100A, 200, 300, 400, 500, 600, 600A, 700 and/or cardiovascular implant devices 100, 100A, 200, 300, 400, 500, 600, 600A, 700 can be expanded by inflation or expansion of another device, e.g., positioned inside the respective cardiovascular implant device. For example, cardiovascular implant devices 100, 100A, 200, 300, 400, 500, 600, 600A, 700 can be compressed such that cardiovascular implant devices 100, 100A, 200, 300, 400, 500, 600, 600A, 700 can fit into a delivery catheter. Cardiovascular implant devices 100, 100A, 200, 300, 400, 500, 600, 600A, 700 can also be made of other materials and can be expandable and collapsible in different ways, e.g., mechanically-expandable, balloon-expandable, self-expandable, or a combination of these. In yet other examples, ones of cardiovascular implant devices 100, 100A, 200, 300, 400, 500, 600, 600A, 700 are not expandable.

### Devices 100 and 100A (FIGS. 5-6)

FIG. 5 is a schematic cross-sectional view of heart H illustrating cardiovascular implant device 100 positioned in inter-atrial septum IS and including curved portion 130. As illustrated in FIG. 5, cardiovascular implant device 100 includes annular body 102, which includes struts 103, central flow tube 104, and flow path 106; and anchoring members 108. Central flow tube 104 includes inflow end 110, outflow end 112, and flow surface 114. Central flow tube 104 further includes straight portion 120 and curved portion 130. FIG. 5 also shows heart H, right atrium RA, left atrium LA, superior vena cava SVC, inferior vena cava IVC, tricuspid valve TV, pulmonary veins PVS, mitral valve MV, inter-atrial septum IS. FIG. 5 further shows right atrial vortical flow RVF, tissue wall plane TWP, tricuspid valve plane TVP, outer diameter OD, axis AX1, and angle α1.

Cardiovascular implant device 100 is an implantable device for use in a cardiovascular system. Cardiovascular implant device 100 is configured to be implanted in blood vessels or chambers of heart H. In the illustrated example, cardiovascular implant device 100 is a flow directing shunt device for shunting blood from one vessel or chamber to another vessel or chamber. Specifically, as shown in FIG. 5, cardiovascular implant device 100 is positioned in inter-atrial septum IS. In other examples, cardiovascular implant device 100 can be positioned in any other tissue wall between adjacent chambers and/or vessels of heart H (or the cardiovascular system). Cardiovascular implant device 100 can be delivered into the cardiovascular system via a catheter (i.e., transcatheter delivery) or can be surgically placed using transcatheter or surgical procedures known in the art.

Annular body 102 is a main body portion of cardiovascular implant device 100. Annular body 102 can be expandable. Annular body 102 is generally cylindrical and is tubular in cross-section but can have a wide variety of different shapes and sizes. Annular body 102 can press against or into tissue walls at the implant site or contour (or extend) around anatomical structures of the cardiovascular system to set and maintain the position of cardiovascular implant device 100. In some examples, e.g., as shown in FIG. 5, annular body 102 can be formed of a plurality of struts 103. Struts 103 can make up a lattice or mesh of annular body 102 and define openings therein. In such examples, annular body 102 can be a stent frame structure for supporting a graft material that guides flow through cardiovascular implant device 100. In other examples, annular body 102 can be solidly formed.

Annular body 102 can be positioned in a puncture in a tissue wall to hold the tissue wall open around annular body 102 so that blood can flow between blood vessels or chambers of heart H through cardiovascular implant device 100. In the example shown in FIG. 5, annular body 102 is positioned in a puncture in inter-atrial septum IS between left atrium LA and right atrium RA so that blood can flow from left atrium LA to right atrium RA through cardiovascular implant device 100. In some examples, struts 103 of annular body 102 form a cage of sorts that is sufficient to hold the tissue wall open around annular body 102. In other examples, a material from which annular body 102 is solidly formed is sufficient to hold the tissue wall open around annular body 102.

Annular body 102 has outer diameter OD. Outer diameter OD is the diameter of annular body 102 as measured to an exterior surface of cardiovascular implant device 100. Outer diameter OD is configured to be approximately the same size as a puncture diameter in a tissue wall in which cardiovascular implant device 100 will be implanted so annular body 102 is able to fit within the puncture. Outer diameter OD can have any size such that cardiovascular implant device 100 is dimensioned to be suitable for a variety of different patient conditions and/or anatomies. In some examples, outer diameter OD can also vary along a length of annular body 102 based on an overall shape or profile of annular body 102.

Annular body 102 includes central flow tube 104, which serves as a conduit for guiding flow through cardiovascular implant device 100. Central flow tube 104 surrounds flow path 106. Flow path 106 is an opening extending through central flow tube 104 such that cardiovascular implant device 100 is open at each opposing end. Flow path 106 is the path through which blood flows or is directed through cardiovascular implant device 100. Central flow tube 104 includes flow surface 114, which is configured to be a flow contacting surface when cardiovascular implant device 100 is implanted in a vessel or chamber of heart H. Flow surface 114 is a radially inner surface of central flow tube 104. Flow path 106 through central flow tube 104 is defined by flow surface 114.

A profile of central flow tube 104 and flow path 106 can be straight, curved, a combination of straight and curved sections, or any other suitable shape, as will be described in greater detail below. In some examples, the profile of central flow tube 104 and flow path 106 can be defined by or the same as a profile of annular body 102 (e.g., as shown in FIG. 5). In other examples, the profile of central flow tube 104 and flow path 106 can be independent of or different from the profile of annular body 102 (e.g., as shown in FIG. 6). Similarly, a cross-sectional shape or profile of central flow tube 104 and annular body 102 can be the same, for example, circular, oval, etc. Alternatively, central flow tube 104 and annular body 102 can have different cross-sectional shapes. For example, annular body 102 could have a circular cross-section and central flow tube 104 could have an oval cross-section. Moreover, the cross-sectional shape of central flow tube 104 and/or annular body can also vary along the length of either. The cross-sectional shape of central flow tube 104 can be selected at various points along its length, such as at outflow end 112, to affect the flow direction.

Central flow tube 104 (and flow path 106 therein) extends from inflow end 110 and outflow end 112. Inflow end 110 can be an end of central flow tube 104 that is relatively upstream of outflow end 112 with respect to a flow of blood through cardiovascular implant device 100, as represented by arrow F in FIG. 5, when cardiovascular implant device 100 is implanted in a blood vessel or chamber of heart H. Accordingly, outflow end 112 is an end of central flow tube 104 that is relatively downstream of inflow end 110 with respect to a flow of blood through cardiovascular implant device 100, as represented by arrow F in FIG. 5, when cardiovascular implant device 100 is implanted in a blood vessel or chamber of heart H. In the example shown in FIG. 5, inflow end 110 is positioned on a left atrial side of inter-atrial septum IS and outflow end 112 is positioned downstream on a right atrial side of inter-atrial septum IS so blood can flow from left atrium LA to right atrium RA through flow path 106. As illustrated in FIG. 5, inflow end 110 can be essentially flush with the left atrial side of inter-atrial septum IS, whereas outflow end 112 can, in some examples, be spaced away from the right atrial side of inter-atrial septum IS within right atrium RA (i.e., cardiovascular implant device 100 can extend further into right atrium RA at outflow end 112 than into left atrium LA at inflow end 110). In other examples, either inflow end 110 or outflow end 112 or both can be flush with or spaced away from the respective side of a tissue wall. Although inflow end 110 is defined as being relatively upstream of outflow end 112, it should be understood that other actual positions of inflow end 110 or outflow end 112 are possible depending on the location where cardiovascular implant device 100 is implanted. Central flow tube 104 can have any suitable length as measured from inflow end 110 to outflow end 112. For example, central flow tube 104 can be designed to have a length that approximates the thickness of inter-atrial septum IS or another tissue wall in which cardiovascular implant device 100 is positioned. In other examples, central flow tube 104 can be longer or shorter than a thickness of inter-atrial septum IS or another tissue wall.

In general, central flow tube 104 can be formed of any suitable material for forming a tubular structure that surrounds flow path 106. For example, all or a portion of central flow tube 104 can be formed of a graft material. The graft material can be a synthetic material, such as a woven polyester or a polytetrafluoroethylene (PTFE), a biologic material, a metallic material, or other materials, to name a few non-limiting examples. Central flow tube 104 formed of a graft material can be supported in cardiovascular implant device 100 by struts 103 of annular body 102. In such examples, central flow tube 104 can be attached to struts 103 of annular body 102 by any suitable attachment means, such as by stitching, gluing, tying, etc. In other examples, central flow tube 104 can be solidly formed with annular body 104.

One or more anchoring members 108 extend outward from annular body 102. Anchoring members 108 hold cardiovascular implant device 100 in position in a tissue wall when cardiovascular implant device 100 is implanted in the body. Anchoring members 108 can take any suitable form for securing cardiovascular implant device 100 to a tissue wall. In some examples, anchoring members 108 can be one or more arms. In other examples, anchoring member 108 can be a flange or annular lip that is configured to have a larger diameter than a diameter of the puncture or opening in which cardiovascular implant device 100 is positioned such that cardiovascular implant device 100 is prevented from slipping through the puncture or opening. In some examples, anchoring members 108 can be curved toward the tissue wall or, alternatively, can rest flush against the tissue wall. As illustrated in FIG. 5, cardiovascular implant device 100 can include one or more anchoring members 108 extending from one end of central flow tube 104. Specifically, cardiovascular implant device 100 can include anchoring members 108 adjacent inflow end 110. In other examples, cardiovascular implant device 100 can include anchoring members 108 adjacent outflow end 112. In yet other examples, cardiovascular implant device 100 can include anchoring members 108 at both inflow end 110 and outflow end 112.

As illustrated in FIG. 5, central flow tube 104 includes straight portion 120 and curved portion 130. Straight portion 120 is a first portion or segment of central flow tube 104. In the example shown in FIG. 5, straight portion 120 is adjacent to and extends from inflow end 110 to capture blood flowing into cardiovascular implant device 100. A length of straight portion 120 is sized to span a puncture in a tissue wall within which cardiovascular implant device 100 is configured to be positioned. Curved portion 130 is a second portion or segment of central flow tube 104. Curved portion 130 is connected to straight portion 120. In the example, shown in FIG. 5, curved portion 130 is adjacent to and extends from outflow end 112 to straight portion 120. That is, curved portion 130 is a relatively downstream portion of central flow tube 104 and straight portion 120 is a relatively upstream portion of central flow tube 104 with respect to a direction of blood flow through cardiovascular implant device 100 when implanted in a tissue wall. Curved portion 130 can be continuous with straight portion 120. Curved portion 130 and straight portion 120 are illustrated in FIG. 5 as having similar lengths; however, it should be understood that curved portion 130 and straight portion 120 can have any relative lengths with respect to each other.

Curved portion 130 is a flow directing component of cardiovascular implant device 100. Curved portion 130 is positioned to direct the flow of blood out of cardiovascular implant device 100 in a particular direction. More specifically, curved portion 130 is curved to direct the flow of blood out of cardiovascular implant device 100 in a particular direction. As illustrated in FIG. 5, curved portion 130 is configured by the positioning of cardiovascular implant device 100 to curve toward tricuspid valve plane TVP (a plane that includes the annulus of tricuspid valve TV). Accordingly, curved portion 130 is configured to direct the flow out of cardiovascular implant device 100 toward tricuspid valve plane TVP. Curved portion 130 defines a turn in flow path 106. When cardiovascular implant device 100 is implanted in inter-atrial septum IS, the turn aligns the portion of flow path 106 at outflow end 112 with the natural flow pattern in right atrium RA. Axis AX1 drawn longitudinally through outflow end 112 (which approximates a longitudinal axis aligned with the flow of blood out of central flow tube 104) forms angle α1 with tissue wall plane TWP of the tissue wall (e.g., inter-atrial septum IS) in which cardiovascular implant device 100 is configured to be positioned. Tissue wall plane TWP is a vertical reference plane defined by the tissue wall and so will be approximately perpendicular to flow path 106 where it crosses the tissue wall. In some examples, angle α1 is between zero and seventy-five degrees (0°-75°).

Because curved portion 130 is adjacent outflow end 112, curved portion 130 is configured to face or extend partially into right atrium RA when cardiovascular implant device 100 is implanted in inter-atrial septum IS. The projection of curved portion 130 into right atrium RA can be minimized so that curved portion 130 only projects into right atrium RA sufficient to fix cardiovascular implant device 100 in place in inter-atrial septum IS.

Once cardiovascular implant device 100 is implanted in cardiovascular system (e.g., inter-atrial septum IS as shown in FIG. 5 or another tissue wall), circulating blood passes through flow path 106 of cardiovascular implant device 100. In the example shown in FIG. 5, blood flows from left atrium LA, through flow path 106, and into right atrium RA. As blood flows out of cardiovascular implant device 100, curved portion 130 aligns the flow of blood with a natural flow pattern of blood in right atrium RA so that the flow of blood out of cardiovascular implant device 100 joins with the natural flow pattern of blood in right atrium RA. More specifically, curved portion 130 aligns the flow of blood out of cardiovascular implant device 100 with a natural vortical flow pattern of blood (i.e., the right-sided flow vortex) in right atrium RA (indicated in FIG. 5 by the schematic stream lines labeled RVF). As illustrated by arrow F in FIG. 5, the flow of blood out of cardiovascular implant device 100 is directed in a curved path along the right atrial side of inter-atrial septum IS and toward tricuspid valve plane TVP, rather than jetting across right atrium RA and dissecting or otherwise disrupting the natural vortical flow pattern. In this way, the flow of blood out of cardiovascular implant device 100 can join with blood flowing down along inter-atrial septum IS and merge into the right atrial vortex.

Cardiovascular implant device 100, including curved portion 130, can minimize disruption to or potentially enhance the natural flow patterns local to a site where cardiovascular implant device 100 is implanted in heart H. When cardiovascular implant device 100 is implanted in inter-atrial septum IS, blood flowing from left atrium LA to right atrium RA through cardiovascular implant device 100 can be less disruptive to the natural rotational (e.g., vortical) flow pattern in right atrium RA because—in contrast to traditional septal shunt devices which may cause blood flow to jet across the right atrium—the flow out of cardiovascular implant device 100 is aligned with the natural vortical flow pattern. Utilizing curved portion 130 to align the flow out of cardiovascular implant device 100 with the natural flow pattern in a chamber or vessel of heart H minimizes any disruptions to the natural flow pattern that could otherwise result from implantation of a traditional shunt device with no directional component. Further, aligning the flow out of cardiovascular implant device 100 could also potentially mitigate reduced flow due to a pathophysiology or other cause or enhance baseline flow. As a result, cardiovascular implant device 100 can maintain kinetic energy of the cardiovascular blood flow, which in turn reduces the cardiac work needed and improves cardiac efficiency. These hemodynamic effects can potentially improve patient outcomes after receiving cardiovascular implant device 100 because cardiovascular implant device 100 can be more effective and potentially safer.

FIG. 6 is a schematic cross-sectional view of inter-atrial septum IS illustrating cardiovascular implant device 100A positioned in inter-atrial septum IS and including internal curved portion 130A. As illustrated in FIG. 6, cardiovascular implant device 100A includes annular body 102A, which includes struts 103A, central flow tube 104A and flow path 106A; and anchoring members 108A. Central flow tube 104A includes inflow end 110A, outflow end 112A, and flow surface 114A. Central flow tube 104A further includes straight portion 120A and curved portion 130A. FIG. 6 also shows right atrium RA, left atrium LA, and inter-atrial septum IS. FIG. 6 further shows tissue wall plane TWP, outer diameter OD, axis AX1, and angle α1.

Cardiovascular implant device 100A has a generally similar structure, design, and function to cardiovascular implant device 100 described above with reference to FIG. 5, except cardiovascular implant device 100A includes internal curved portion 130A. Compared to curved portion 130 shown in FIG. 5, curved portion 130A is formed interiorly in annular body 102 such that curved portion 130A and outflow end 112A do not extend significantly, if at all, beyond the tissue wall in which cardiovascular implant device 100A is implanted. As such, cardiovascular implant device 100A includes anchoring members 108 adjacent both inflow end 110A and outflow end 112A. As illustrated in FIG. 6, central flow tube 104A and annular body 102A do not have the same profile. Central flow tube 104A has a curved profile though curved portion 130A, but annular body 102 has a straight profile. In other words, the curvature of curved portion 130A is not reflected by outer diameter OD.

### Device 200 (FIG. 7)

FIG. 7 is a schematic cross-sectional view of heart H illustrating cardiovascular implant device 200 positioned in inter-atrial septum IS and including flap 240. As illustrated in FIG. 7, cardiovascular implant device 200 includes annular body 202, which includes struts 203, central flow tube 204, and flow path 206; and anchoring members 208. Central flow tube 204 includes inflow end 210, outflow end 212, and flow surface 214. Cardiovascular implant device 200 further includes flap 240, flexible joint 242, and stopper 244. FIG. 7 also shows heart H, right atrium RA, left atrium LA, superior vena cava SVC, inferior vena cava IVC, tricuspid valve TV, pulmonary veins PVS, mitral valve MV, inter-atrial septum IS. FIG. 7 further shows right atrial vortical flow RVF, tissue wall plane TWP, tricuspid valve plane TVP, outer diameter OD, axis AX2, axis LX2, and angle α2.

Cardiovascular implant device 200 is an implantable device for use in a cardiovascular system. Cardiovascular implant device 200 is configured to be implanted in blood vessels or chambers of heart H. In the illustrated example, cardiovascular implant device 200 is a flow directing shunt device for shunting blood from one vessel or chamber to another vessel or chamber. Specifically, as shown in FIG. 7, cardiovascular implant device 200 is positioned in inter-atrial septum IS. In other examples, cardiovascular implant device 200 can be positioned in any other tissue wall between adjacent chambers and/or vessels of heart H (or the cardiovascular system). Cardiovascular implant device 200 can be delivered into the cardiovascular system via a catheter (i.e., transcatheter delivery) or can be surgically placed using transcatheter or surgical procedures known in the art.

Annular body 202 is a main body portion of cardiovascular implant device 200. Annular body 202 can be expandable. Annular body 202 is generally cylindrical and is tubular in cross-section but can have a wide variety of different shapes and sizes. Annular body 202 can press against or into tissue walls at the implant site or contour (or extend) around anatomical structures of the cardiovascular system to set and maintain the position of cardiovascular implant device 200. In some examples, e.g., as shown in FIG. 7, annular body 202 can be formed of a plurality of struts 203. Struts 203 can make up a lattice or mesh of annular body 202 and define openings therein. In such examples, annular body 202 can be a stent frame structure for supporting a graft material that guides flow through cardiovascular implant device 200. In other examples, annular body 202 can be solidly formed.

Annular body 202 can be positioned in a puncture in a tissue wall to hold the tissue wall open around annular body 202 so that blood can flow between blood vessels or chambers of heart H through cardiovascular implant device 200. In the example shown in FIG. 7, annular body 202 is positioned in a puncture in inter-atrial septum IS between left atrium LA and right atrium RA so that blood can flow from left atrium LA to right atrium RA through cardiovascular implant device 200. In some examples, struts 203 of annular body 202 form a cage of sorts that is sufficient to hold the tissue wall open around annular body 202. In other examples, a material from which annular body 202 is solidly formed is sufficient to hold the tissue wall open around annular body 202.

Annular body 202 has outer diameter OD. Outer diameter OD is the diameter of annular body 202 as measured to an exterior surface of cardiovascular implant device 200. Outer diameter OD is configured to be approximately the same size as a puncture diameter in a tissue wall in which cardiovascular implant device 200 will be implanted so annular body 202 is able to fit within the puncture. Outer diameter OD can have any size such that cardiovascular implant device 200 is dimensioned to be suitable for a variety of different patient conditions and/or anatomies. In some examples, outer diameter OD can also vary along a length of annular body 202 based on an overall shape or profile of annular body 202.

Annular body 202 includes central flow tube 204, which serves as a conduit for guiding flow through cardiovascular implant device 200. Central flow tube 204 surrounds flow path 206. Flow path 206 is an opening extending through central flow tube 204 such that cardiovascular implant device 200 is open at each opposing end. Flow path 206 is the path through which blood flows or is directed through cardiovascular implant device 200. Central flow tube 204 includes flow surface 214, which is configured to be a flow contacting surface when cardiovascular implant device 200 is implanted in a vessel or chamber of heart H. Flow surface 214 is a radially inner surface of central flow tube 204. Flow path 206 through central flow tube 204 is defined by flow surface 214.

A profile of central flow tube 204 and flow path 206 can be straight, curved, a combination of straight and curved sections, or any other suitable shape. In some examples, the profile of central flow tube 204 and flow path 206 can be defined by or the same as a profile of annular body 202 (e.g., as shown in FIG. 5). In other examples, the profile of central flow tube 204 and flow path 206 can be independent of or different from the profile of annular body 202 (e.g., as shown in FIG. 6). Similarly, a cross-sectional shape or profile of central flow tube 204 and annular body 202 can be the same, for example, circular, oval, etc. Alternatively, central flow tube 204 and annular body 202 can have different cross-sectional shapes. For example, annular body 202 could have a circular cross-section and central flow tube 204 could have an oval cross-section. Moreover, the cross-sectional shape of central flow tube 204 and/or annular body can also vary along the length of either. The cross-sectional shape of central flow tube 204 can be selected at various points along its length, such as at outflow end 212, to affect the flow direction.

Central flow tube 204 (and flow path 206 therein) extends from inflow end 210 and outflow end 212. Inflow end 210 can be an end of central flow tube 204 that is relatively upstream of outflow end 212 with respect to a flow of blood through cardiovascular implant device 200, as represented by arrow F in FIG. 7, when cardiovascular implant device 200 is implanted in a blood vessel or chamber of heart H. Accordingly, outflow end 212 is an end of central flow tube 204 that is relatively downstream of inflow end 210 with respect to a flow of blood through cardiovascular implant device 200, as represented by arrow F in FIG. 7, when cardiovascular implant device 200 is implanted in a blood vessel or chamber of heart H. In the example shown in FIG. 7, inflow end 210 is positioned on a left atrial side of inter-atrial septum IS and outflow end 212 is positioned downstream on a right atrial side of inter-atrial septum IS so blood can flow from left atrium LA to right atrium RA through flow path 206. As illustrated in FIG. 7, inflow end 210 can be essentially flush with the left atrial side of inter-atrial septum IS, whereas outflow end 212 can, in some examples, be spaced away from the right atrial side of inter-atrial septum IS within right atrium RA (i.e., cardiovascular implant device 200 can extend further into right atrium RA at outflow end 212 than into left atrium LA at inflow end 210). In other examples, either inflow end 210 or outflow end 212 or both can be flush with or spaced away from the respective side of a tissue wall. Although inflow end 210 is defined as being relatively upstream of outflow end 212, it should be understood that other actual positions of inflow end 210 or outflow end 212 are possible depending on the location where cardiovascular implant device 200 is implanted. Central flow tube 204 can have any suitable length as measured from inflow end 210 to outflow end 212. For example, central flow tube 204 can be designed to have a length that approximates the thickness of inter-atrial septum IS or another tissue wall in which cardiovascular implant device 200 is positioned. In other examples, central flow tube 204 can be longer or shorter than a thickness of inter-atrial septum IS or another tissue wall.

In general, central flow tube 204 can be formed of any suitable material for forming a tubular structure that surrounds flow path 206. For example, all or a portion of central flow tube 204 can be formed of a graft material. The graft material can be a synthetic material, such as a woven polyester or a polytetrafluoroethylene (PTFE), a biologic material, a metallic material, or other materials, to name a few non-limiting examples. Central flow tube 204 formed of a graft material can be supported in cardiovascular implant device 200 by struts 203 of annular body 202. In such examples, central flow tube 204 can be attached to struts 203 of annular body 202 by any suitable attachment means, such as by stitching, gluing, tying, etc. In other examples, central flow tube 204 can be solidly formed with annular body 204.

One or more anchoring members 208 extend outward from annular body 202. Anchoring members 208 hold cardiovascular implant device 200 in position in a tissue wall when cardiovascular implant device 200 is implanted in the body. Anchoring members 208 can take any suitable form for securing cardiovascular implant device 200 to a tissue wall. In some examples, anchoring members 208 can be one or more arms. In other examples, anchoring member 208 can be a flange or annular lip that is configured to have a larger diameter than a diameter of the puncture or opening in which cardiovascular implant device 200 is positioned such that cardiovascular implant device 200 is prevented from slipping through the puncture or opening. In some examples, anchoring members 208 can be curved toward the tissue wall or, alternatively, can rest flush against the tissue wall. As illustrated in FIG. 7, cardiovascular implant device 200 can include one or more anchoring members 208 extending from one end of central flow tube 204. Specifically, cardiovascular implant device 200 can include anchoring members 208 adjacent inflow end 210. In other examples, cardiovascular implant device 200 can include anchoring members 208 adjacent outflow end 212. In yet other examples, cardiovascular implant device 200 can include anchoring members 208 at both inflow end 210 and outflow end 212.

As illustrated in FIG. 7, cardiovascular implant device 200 includes flap 240. Flap 240 is connected to annular body 202 adjacent outflow end 212 of central flow tube 204. In some examples, flap 240 is sized and shaped like a door or cover that would fit over an end of cardiovascular implant device 200. In other examples, flap 240 can have any suitable size and shape. In some examples, flap 240 is solidly formed of a relatively flexible but impermeable material. In other examples, a perimeter of flap 240 is formed of a wire frame and an impermeable cloth is stretched over the wire frame. For example, the wire frame can be formed of nitinol (a nickel titanium alloy) or a similar shape-memory material. In yet other examples, all or a portion of flap 240 can be formed of a biologic material, such as pericardium.

Flap 240 is connected to annular body 202 by flexible joint 242. Flap 240 is positionable with respect to annular body 202 at flexible joint 242. That is, flap 240 can pivot at flexible joint 242 to be positioned at different angles. In some examples, flexible joint 242 is a hinge. In other examples, flexible joint 242 is a flexible piece of material that connects or extends between flap 240 and annular body 202. In yet other examples, flexible joint 242 can be any suitable flexible attachment mechanism. Cardiovascular implant device 200 can also include stopper 244 adjacent flexible joint 242 for preventing flap 240 from moving beyond a maximum opening angle. For example, stopper 244 can prevent flap 240 from flipping fully open (moving) 180° when there is high pressure flow through cardiovascular implant device 200.

Flap 240 is a flow directing component of cardiovascular implant device 200. Flap 240 is positioned or positionable to direct the flow of blood out of cardiovascular implant device 200 in a particular direction. More specifically, flap 240 is angled to direct the flow of blood out of cardiovascular implant device 200 in a particular direction. Flap 240 is positioned such that it is angled toward longitudinal axis LX2 through central flow tube 204 (i.e., axis AX2 of flap 240 intersects axis LX2). As illustrated in FIG. 7, flap 240 is configured by the positioning of cardiovascular implant device 200 to be angled toward tricuspid valve plane TVP (a plane that includes the annulus of tricuspid valve TV) and, accordingly, to direct the flow of blood out of cardiovascular implant device 200 toward tricuspid valve plane TVP. Similar to curved portion 130 shown in FIG. 5, flap 240 blocks a direction of blood flow out of cardiovascular implant device 200 so that the blood flow must turn. When cardiovascular implant device 200 is implanted in inter-atrial septum IS, flap 240 is positioned to align the flow of blood out of cardiovascular implant device 200 with the natural flow pattern in right atrium RA. Axis AX2 of flap 240 (which is approximately parallel to the flow path of blood out of central flow tube 204 and so can be used to approximate a longitudinal axis aligned with the flow of blood out of central flow tube 204) forms angle α2 with tissue wall plane TWP of the tissue wall (e.g., inter-atrial septum IS) in which cardiovascular implant device 200 is configured to be positioned. Tissue wall plane TWP is a vertical reference plane defined by the tissue wall and so will be approximately perpendicular to flow path 206 where it crosses the tissue wall. In some examples, angle α2 is between zero and seventy-five degrees (0°-75°).

Because flap 240 is attached to annular body 202 adjacent outflow end 212, flap 240 is configured to extend partially into right atrium RA when cardiovascular implant device 200 is implanted in inter-atrial septum IS. Flap 240 extends at angle α2 with respect to tissue wall plane TWP. In some examples, flap 240 is biased open at angle α2 when attached to annular body 202 to promote flow through and out of cardiovascular implant device 200. In other examples, flap 240 can be configured to open and close to some degree based on the pressure difference between left atrium LA and right atrium RA. In such examples, when there is a greater pressure difference between left atrium LA and right atrium RA, flow through cardiovascular implant device 200 can force flap 240 to open more and greater flow will be able to pass through cardiovascular implant device 200. Flap 240 can be configured such that angle α2 is a maximum opening angle based on desired flow characteristics out of cardiovascular implant device 200. The maximum opening angle is set by stopper 244.

Once cardiovascular implant device 200 is implanted in cardiovascular system (e.g., inter-atrial septum IS as shown in FIG. 7 or another tissue wall), circulating blood passes through flow path 206 of cardiovascular implant device 200. In the example shown in FIG. 7, blood flows from left atrium LA, through flow path 206, and into right atrium RA. As blood flows out of cardiovascular implant device 200, flap 240 aligns the flow of blood with a natural flow pattern of blood in right atrium RA so that the flow of blood out of cardiovascular implant device 200 joins with the natural flow pattern of blood in right atrium RA. More specifically, flap 240 aligns the flow of blood out of cardiovascular implant device 200 with a natural vortical flow pattern of blood (i.e., the right-sided flow vortex) in right atrium RA (indicated in FIG. 7 by the schematic stream lines labeled RVF). As illustrated by arrow F in FIG. 7, the flow of blood out of cardiovascular implant device 200 is directed in a curved path along the right atrial side of inter-atrial septum IS and toward tricuspid valve plane TVP, rather than jetting across right atrium RA and dissecting or otherwise disrupting the natural vortical flow pattern. In this way, the flow of blood out of cardiovascular implant device 200 can join with blood flowing down along inter-atrial septum IS and merge into the right atrial vortex.

Cardiovascular implant device 200, including flap 240, can minimize disruption to or potentially enhance the natural flow patterns local to a site where cardiovascular implant device 200 is implanted in heart H. When cardiovascular implant device 200 is implanted in inter-atrial septum IS, blood flowing from left atrium LA to right atrium RA through cardiovascular implant device 200 can be less disruptive to the natural rotational (e.g., vortical) flow pattern in right atrium RA because—in contrast to traditional septal shunt devices which may cause blood flow to jet across the right atrium—the flow out of cardiovascular implant device 200 is aligned with the natural vortical flow pattern. Utilizing flap 240 to align the flow out of cardiovascular implant device 200 with the natural flow pattern in a chamber or vessel of heart H minimizes any disruptions to the natural flow pattern that could otherwise result from implantation of a traditional shunt device with no directional component. Further, aligning the flow out of cardiovascular implant device 200 could also potentially mitigate reduced flow due to a pathophysiology or other cause or enhance baseline flow. As a result, cardiovascular implant device 200 can maintain kinetic energy of the cardiovascular blood flow, which in turn reduces the cardiac work needed and improves cardiac efficiency. These hemodynamic effects can potentially improve patient outcomes after receiving cardiovascular implant device 200 because cardiovascular implant device 200 can be more effective and potentially safer.

### Device 300 (FIGS. 8A-8B)

FIG. 8A is a schematic cross-sectional view of heart H illustrating cardiovascular implant device 300 positioned adjacent shunt S in inter-atrial septum IS. FIG. 8B is an enlarged side view of cardiovascular implant device 300. FIGS. 8A and 8B will be discussed together. As illustrated in FIGS. 8A-8B, cardiovascular implant device 300 includes anchoring member 335, flap 340, flexible joint 342, and stopper 344. FIG. 8A also shows heart H, right atrium RA, left atrium LA, superior vena cava SVC, inferior vena cava IVC, tricuspid valve TV, pulmonary veins PVS, mitral valve MV, inter-atrial septum IS. FIG. 8A further shows shunt S, flow path SFP, right atrial vortical flow RVF, tissue wall plane TWP, tricuspid valve plane TVP, axis AX3, axis LX3, and angle α3.

Cardiovascular implant device 300 is an implantable device for use in a cardiovascular system. Cardiovascular implant device 300 is configured to be implanted in blood vessels or chambers of heart H. In the illustrated example, cardiovascular implant device 300 is a flow directing device that is independent of a shunt device, stent device, or other implantable device. Specifically, as shown in FIG. 8A, cardiovascular implant device 300 is positioned adjacent shunt S in inter-atrial septum IS. In other examples, cardiovascular implant device 300 can be positioned adjacent a puncture or opening in any other tissue wall between adjacent chambers and/or vessels of heart H (or the cardiovascular system). Shunt S is a shunt (non-implant) between left atrium LA and right atrium RA that is formed by excision of tissue from inter-atrial septum IS to create an opening. In some examples, the tissue can be excised to create shunt S with the application of radio frequency (RF) energy or other energy. Cardiovascular implant device 300 can be delivered into the cardiovascular system via a catheter (i.e., transcatheter delivery) or can be surgically placed using transcatheter or surgical procedures known in the art.

As illustrated in FIG. 8A, shunt flow path SFP is an opening extending through the tissue wall where shunt S is formed such that shunt S is open at each opposing end. Shunt flow path SFP is the path through which blood flows or is directed through shunt S. In the example shown in FIG. 8A, shunt flow path SFP spans inter-atrial septum IS and extends from left atrium LA to right atrium RA.

As illustrated in FIGS. 8A-8B, cardiovascular implant device 300 includes flap 340. Flap 340 is connected to anchoring member 335. In some examples, flap 340 is sized and shaped like a door or cover that would fit over one end of shunt S. In other examples, flap 340 can have any suitable size and shape. In some examples, flap 340 is solidly formed of a relatively flexible but impermeable material. In other examples, a perimeter of flap 340 is formed of a wire frame and an impermeable cloth is stretched over the wire frame. For example, the wire frame can be formed of nitinol (a nickel titanium alloy) or a similar shape-memory material. In yet other examples, all or a portion of flap 340 can be formed of a biologic material, such as pericardium.

Anchoring member 335 is configured to secure cardiovascular implant device 300, including flap 340, to a tissue wall. Anchoring member 335 can further include fasteners or other attachment mechanisms for anchoring cardiovascular implant device 300 to the tissue wall. In the example shown in FIG. 8A, anchoring member 335 secures cardiovascular implant device 300 to a right atrial side of inter-atrial septum IS. Anchoring member 335 is positioned adjacent to shunt S. More specifically, anchoring member 335 is positioned above shunt S with respect to the orientation of heart H when a human is upright so that flap 340 extends downward over shunt S. In other examples, anchoring member 335 could be positioned in any location around shunt S.

Flap 340 is connected to anchoring member 335 by flexible joint 342. Flap 340 is positionable with respect to shunt S at flexible joint 342. That is, flap 340 can pivot at flexible joint 342 to be positioned at different angles. In some examples, flexible joint 342 is a hinge. In other examples, flexible joint 342 is a flexible piece of material that connects or extends between flap 340 and anchoring member 335. In yet other examples, flexible joint 342 can be any suitable flexible attachment mechanism. Cardiovascular implant device 300 can also include stopper 344 adjacent flexible joint 342 for preventing flap 340 from moving beyond a maximum opening angle. For example, stopper 344 can prevent flap 340 from flipping fully open (moving) 180° when there is high pressure flow through shunt S.

Flap 340 is a flow directing component of cardiovascular implant device 300. Flap 340 is positioned or positionable to direct the flow of blood out of shunt S in a particular direction. More specifically, flap 340 is angled to direct the flow of blood out of shunt S in a particular direction. Flap 340 is positioned such that it is angled toward longitudinal axis LX3 through shunt S (i.e., axis AX3 of flap 340 intersects axis LX3). As illustrated in FIG. 8A, flap 340 is configured by the positioning of cardiovascular implant device 300 to be angled toward tricuspid valve plane TVP (a plane that includes the annulus of tricuspid valve TV) and, accordingly, to direct the flow of blood out of shunt S toward tricuspid valve plane TVP. Flap 340 blocks a direction of blood flow out of shunt S so that the blood flow must turn. When cardiovascular implant device 300 is implanted in inter-atrial septum IS, flap 340 is positioned to align the flow of blood out of shunt S with the natural flow pattern in right atrium RA. Axis AX3 of flap 340 (which is approximately parallel to the flow path of blood out of shunt S and so can be used to approximate a longitudinal axis aligned with the flow of blood out of shunt S) forms angle α3 with tissue wall plane TWP of the tissue wall (e.g., inter-atrial septum IS) to which cardiovascular implant device 300 is configured to be attached. Tissue wall plane TWP is a vertical reference plane defined by the tissue wall and so will be approximately perpendicular to shunt flow path SFP where it crosses the tissue wall. In some examples, angle α3 is between zero and seventy-five degrees (0°-75°).

Because flap 340 is attached to anchoring member 335, flap 340 is configured to extend partially into right atrium RA when cardiovascular implant device 300 is attached to a right atrial side of inter-atrial septum IS. Flap 340 extends at angle α3 with respect to tissue wall plane TWP. In some examples, flap 340 is biased open at angle α3 when attached to anchoring member 335 to promote flow through and out of shunt S. In other examples, flap 340 can be configured to open and close to some degree based on the pressure difference between left atrium LA and right atrium RA. In such examples, when there is a greater pressure difference between left atrium LA and right atrium RA, flow through shunt S can force flap 340 to open more and greater flow will be able to pass through shunt S. Flap 340 can be configured such that angle α3 is a maximum opening angle based on desired flow characteristics out of shunt S. The maximum opening angle is set by stopper 344.

Once cardiovascular implant device 300 is implanted in cardiovascular system (e.g., inter-atrial septum IS as shown in FIG. 8A or another tissue wall), circulating blood passes through flow path SFP of shunt S. In the example shown in FIG. 8A, blood flows from left atrium LA, through flow path SFP, and into right atrium RA. As blood flows out of shunt S, flap 340 aligns the flow of blood with a natural flow pattern of blood in right atrium RA so that the flow of blood out of shunt S joins with the natural flow pattern of blood in right atrium RA. More specifically, flap 340 aligns the flow of blood out of shunt S with a natural vortical flow pattern of blood (i.e., the right-sided flow vortex) in right atrium RA (indicated in FIG. 8A by the schematic stream lines labeled RVF). As illustrated by arrow F in FIG. 8A, the flow of blood out of shunt S is directed in a curved path along the right atrial side of inter-atrial septum IS and toward tricuspid valve plane TVP, rather than jetting across right atrium RA and dissecting or otherwise disrupting the natural vortical flow pattern. In this way, the flow of blood out of shunt S can join with blood flowing down along inter-atrial septum IS and merge into the right atrial vortex.

Cardiovascular implant device 300, including flap 340, can minimize disruption to or potentially enhance the natural flow patterns local to a site where cardiovascular implant device 300 is implanted in heart H. When cardiovascular implant device 300 is implanted in inter-atrial septum IS, blood flowing from left atrium LA to right atrium RA through shunt S can be less disruptive to the natural rotational (e.g., vortical) flow pattern in right atrium RA because—in contrast to traditional septal shunt devices which may cause blood flow to jet across the right atrium—the flow out of shunt S is aligned with the natural vortical flow pattern. Utilizing flap 340 to align the flow out of shunt S with the natural flow pattern in a chamber or vessel of heart H minimizes any disruptions to the natural flow pattern that could otherwise result from implantation of a traditional shunt device with no directional component. Further, aligning the flow out of shunt S could also potentially mitigate reduced flow due to a pathophysiology or other cause or enhance baseline flow. As a result, cardiovascular implant device 300 can maintain kinetic energy of the cardiovascular blood flow, which in turn reduces the cardiac work needed and improves cardiac efficiency. These hemodynamic effects can potentially improve patient outcomes after receiving cardiovascular implant device 300 because cardiovascular implant device 300 can be more effective and potentially safer.

Further, as will be apparent from FIGS. 7-8B together, a flap, such as flap 240 or flap 340, can be utilized either as a modification to a shunt device (e.g., cardiovascular implant device 200) or as a standalone flow directing feature (e.g., cardiovascular implant device 300). This makes flaps 240, 340 versatile features that can be used both for patients who will undergo a procedure to have a shunt device placed and patients who instead have a non-device shunt procedure.

### Device 400 (FIG. 9)

FIG. 9 is a schematic cross-sectional view of heart H illustrating cardiovascular implant device 400 positioned in inter-atrial septum IS and including internal guide wall 450. As illustrated in FIG. 9, cardiovascular implant device 400 includes annular body 402, which includes struts 403, central flow tube 404, and flow path 406; and anchoring members 408. Central flow tube 404 includes inflow end 410, outflow end 412, and flow surface 414. Cardiovascular implant device 400 further includes guide wall 450 and spiral flow path 452. FIG. 9 also shows heart H, right atrium RA, left atrium LA, superior vena cava SVC, inferior vena cava IVC, tricuspid valve TV, pulmonary veins PVS, mitral valve MV, inter-atrial septum IS. FIG. 9 further shows right atrial vortical flow RVF, tissue wall plane TWP, tricuspid valve plane TVP, outer diameter OD, axis AX4, and angle α4.

Cardiovascular implant device 400 is an implantable device for use in a cardiovascular system. Cardiovascular implant device 400 is configured to be implanted in blood vessels or chambers of heart H. In the illustrated example, cardiovascular implant device 400 is a flow directing shunt device for shunting blood from one vessel or chamber to another vessel or chamber. Specifically, as shown in FIG. 9, cardiovascular implant device 400 is positioned in inter-atrial septum IS. In other examples, cardiovascular implant device 400 can be positioned in any other tissue wall between adjacent chambers and/or vessels of heart H (or the cardiovascular system). Cardiovascular implant device 400 can be delivered into the cardiovascular system via a catheter (i.e., transcatheter delivery) or can be surgically placed using transcatheter or surgical procedures known in the art.

Annular body 402 is a main body portion of cardiovascular implant device 400. Annular body 402 can be expandable. Annular body 402 is generally cylindrical and is tubular in cross-section but can have a wide variety of different shapes and sizes. Annular body 402 can press against or into tissue walls at the implant site or contour (or extend) around anatomical structures of the cardiovascular system to set and maintain the position of cardiovascular implant device 400. In some examples, e.g., as shown in FIG. 9, annular body 402 can be formed of a plurality of struts 403. Struts 403 can make up a lattice or mesh of annular body 402 and define openings therein. In such examples, annular body 402 can be a stent frame structure for supporting a graft material that guides flow through cardiovascular implant device 400. In other examples, annular body 402 can be solidly formed.

Annular body 402 can be positioned in a puncture in a tissue wall to hold the tissue wall open around annular body 402 so that blood can flow between blood vessels or chambers of heart H through cardiovascular implant device 400. In the example shown in FIG. 9, annular body 402 is positioned in a puncture in inter-atrial septum IS between left atrium LA and right atrium RA so that blood can flow from left atrium LA to right atrium RA through cardiovascular implant device 400. In some examples, struts 403 of annular body 402 form a cage of sorts that is sufficient to hold the tissue wall open around annular body 402. In other examples, a material from which annular body 402 is solidly formed is sufficient to hold the tissue wall open around annular body 402.

Annular body 402 has outer diameter OD. Outer diameter OD is the diameter of annular body 402 as measured to an exterior surface of cardiovascular implant device 400. Outer diameter OD is configured to be approximately the same size as a puncture diameter in a tissue wall in which cardiovascular implant device 400 will be implanted so annular body 402 is able to fit within the puncture. Outer diameter OD can have any size such that cardiovascular implant device 400 is dimensioned to be suitable for a variety of different patient conditions and/or anatomies. In some examples, outer diameter OD can also vary along a length of annular body 402 based on an overall shape or profile of annular body 402.

Annular body 402 includes central flow tube 404, which serves as a conduit for guiding flow through cardiovascular implant device 400. Central flow tube 404 surrounds flow path 406. Flow path 406 is an opening extending through central flow tube 404 such that cardiovascular implant device 400 is open at each opposing end. Flow path 406 is the path through which blood flows or is directed through cardiovascular implant device 400. Central flow tube 404 includes flow surface 414, which is configured to be a flow contacting surface when cardiovascular implant device 400 is implanted in a vessel or chamber of heart H. Flow surface 414 is a radially inner surface of central flow tube 404. Flow path 406 through central flow tube 404 is defined by flow surface 414.

A profile of central flow tube 404 and flow path 406 can be straight, curved, a combination of straight and curved sections, or any other suitable shape. In some examples, the profile of central flow tube 404 and flow path 406 can be defined by or the same as a profile of annular body 402 (e.g., as shown in FIG. 5). In other examples, the profile of central flow tube 404 and flow path 406 can be independent of or different from the profile of annular body 402 (e.g., as shown in FIG. 6). Similarly, a cross-sectional shape or profile of central flow tube 404 and annular body 402 can be the same, for example, circular, oval, etc. Alternatively, central flow tube 404 and annular body 402 can have different cross-sectional shapes. For example, annular body 402 could have a circular cross-section and central flow tube 404 could have an oval cross-section. Moreover, the cross-sectional shape of central flow tube 404 and/or annular body can also vary along the length of either. The cross-sectional shape of central flow tube 404 can be selected at various points along its length, such as at outflow end 412, to affect the flow direction.

Central flow tube 404 (and flow path 406 therein) extends from inflow end 410 and outflow end 412. Inflow end 410 can be an end of central flow tube 404 that is relatively upstream of outflow end 412 with respect to a flow of blood through cardiovascular implant device 400, as represented by arrow F in FIG. 9, when cardiovascular implant device 400 is implanted in a blood vessel or chamber of heart H. Accordingly, outflow end 412 is an end of central flow tube 404 that is relatively downstream of inflow end 410 with respect to a flow of blood through cardiovascular implant device 400, as represented by arrow F in FIG. 9, when cardiovascular implant device 400 is implanted in a blood vessel or chamber of heart H. In the example shown in FIG. 9, inflow end 410 is positioned on a left atrial side of inter-atrial septum IS and outflow end 412 is positioned downstream on a right atrial side of inter-atrial septum IS so blood can flow from left atrium LA to right atrium RA through flow path 406. As illustrated in FIG. 9, inflow end 410 can be essentially flush with the left atrial side of inter-atrial septum IS, whereas outflow end 412 can, in some examples, be spaced away from the right atrial side of inter-atrial septum IS within right atrium RA (i.e., cardiovascular implant device 400 can extend further into right atrium RA at outflow end 412 than into left atrium LA at inflow end 410). In other examples, either inflow end 410 or outflow end 412 or both can be flush with or spaced away from the respective side of a tissue wall. Although inflow end 410 is defined as being relatively upstream of outflow end 412, it should be understood that other actual positions of inflow end 410 or outflow end 412 are possible depending on the location where cardiovascular implant device 400 is implanted. Central flow tube 404 can have any suitable length as measured from inflow end 410 to outflow end 412. For example, central flow tube 404 can be designed to have a length that approximates the thickness of inter-atrial septum IS or another tissue wall in which cardiovascular implant device 400 is positioned. In other examples, central flow tube 404 can be longer or shorter than a thickness of inter-atrial septum IS or another tissue wall.

In general, central flow tube 404 can be formed of any suitable material for forming a tubular structure that surrounds flow path 406. For example, all or a portion of central flow tube 404 can be formed of a graft material. The graft material can be a synthetic material, such as a woven polyester or a polytetrafluoroethylene (PTFE), a biologic material, a metallic material, or other materials, to name a few non-limiting examples. Central flow tube 404 formed of a graft material can be supported in cardiovascular implant device 400 by struts 403 of annular body 402. In such examples, central flow tube 404 can be attached to struts 403 of annular body 402 by any suitable attachment means, such as by stitching, gluing, tying, etc. In other examples, central flow tube 404 can be solidly formed with annular body 404.

One or more anchoring members 408 extend outward from annular body 402. Anchoring members 408 hold cardiovascular implant device 400 in position in a tissue wall when cardiovascular implant device 400 is implanted in the body. Anchoring members 408 can take any suitable form for securing cardiovascular implant device 400 to a tissue wall. In some examples, anchoring members 408 can be one or more arms. In other examples, anchoring member 408 can be a flange or annular lip that is configured to have a larger diameter than a diameter of the puncture or opening in which cardiovascular implant device 400 is positioned such that cardiovascular implant device 400 is prevented from slipping through the puncture or opening. In some examples, anchoring members 408 can be curved toward the tissue wall or, alternatively, can rest flush against the tissue wall. As illustrated in FIG. 9, cardiovascular implant device 400 can include one or more anchoring members 408 extending from one end of central flow tube 404. Specifically, cardiovascular implant device 400 can include anchoring members 408 adjacent inflow end 410. In other examples, cardiovascular implant device 400 can include anchoring members 408 adjacent outflow end 412. In yet other examples, cardiovascular implant device 400 can include anchoring members 408 at both inflow end 410 and outflow end 412.

As illustrated in FIG. 9, cardiovascular implant device 400 includes guide wall 450. Guide wall 450 is a spiral wall. In some examples, guide wall 450 is helical. Guide wall 450 is connected to flow surface 414 of central flow tube 404 (i.e., a radially inner surface of central flow tube 404). Specifically, guide wall 450 is attached circumferentially to flow surface 414. Because cardiovascular implant device 400 is shown in cross-section in FIG. 9, only a portion (one side or half laterally with respect to central flow tube 404) of guide wall 450 is depicted in FIG. 9, and connecting portions are cut away. Guide wall 450 extends from inflow end 410 to outflow end 412 within an interior of central flow tube 404. As shown schematically in FIG. 9 by the dashed and solid lines within the interior of central flow tube 404, guide wall 450 loops around the interior like a slide from inflow end 410 to outflow end 412. Due to the spiral nature of guide wall 450, flow path 406 extending through central flow tube 404 is also spiral flow path 452. The spiral shape of spiral flow path 452 is defined by the arrangement of guide wall 450 within central flow tube 404.

In general, the structures in the interior of central flow tube 404, including guide wall 450, can be configured to be collapsible or can have physical dimensions sized to avoid interfering with other components if cardiovascular implant device 400 will be delivered with a catheter. Surgical delivery examples may not have the same size-related limitations for implementing guide wall 450.

Guide wall 450 is a flow directing component of cardiovascular implant device 400. More specifically, guide wall 450 is arranged and positioned to guide the flow of blood through central flow tube 404 and to direct the flow of blood out of cardiovascular implant device 400 in a particular direction. Guide wall 450 is positioned to prevent the flow of blood through central flow tube 404 from flowing in a straight path through cardiovascular implant device 400. Instead, blood flowing through central flow tube 404 flows through spiral flow path 452. As illustrated in FIG. 9, guide wall 450 is configured to direct the flow of blood out of cardiovascular implant device 400 toward tricuspid valve plane TVP (a plane that includes the annulus of tricuspid valve TV). When cardiovascular implant device 400 is implanted in inter-atrial septum IS, guide wall 450 is positioned to align the flow of blood out of cardiovascular implant device 400 with the natural flow pattern in right atrium RA. As illustrated in FIG. 9, axis AX4 is a longitudinal axis aligned with the flow of blood out of central flow tube 404. Axis AX4 forms angle α4 with tissue wall plane TWP of the tissue wall (e.g., inter-atrial septum IS) in which cardiovascular implant device 400 is configured to be positioned. Tissue wall plane TWP is a vertical reference plane defined by the tissue wall and so will be approximately perpendicular to flow path 406 where it crosses the tissue wall. Angle α4 can be considered an exit angle of the blood flowing out of cardiovascular implant device 400. In some examples, angle α4 is between zero and seventy-five degrees (0°-75°).

Once cardiovascular implant device 400 is implanted in cardiovascular system (e.g., inter-atrial septum IS as shown in FIG. 9 or another tissue wall), circulating blood passes through flow path 406 of cardiovascular implant device 400. In the example shown in FIG. 9, blood flows from left atrium LA, through flow path 406 (spiral flow path 452), and into right atrium RA. As blood flows through cardiovascular implant device 400, guide wall 450 guides the blood flow so that the flow of blood out of cardiovascular implant device 400 aligns and joins with a natural flow pattern of blood in right atrium RA. Guide wall 450 can also impart rotational velocity to the flow of blood through flow path 406 to help align the flow. More specifically, guide wall 450 guides the flow of blood through cardiovascular implant device 400 to align and join the flow of blood out of cardiovascular implant device 400 with a natural vortical flow pattern of blood (i.e., the right-sided flow vortex) in right atrium RA (indicated in FIG. 9 by the schematic stream lines labeled RVF). As illustrated by arrow F in FIG. 9, the flow of blood out of cardiovascular implant device 400 is directed in a curved path along the right atrial side of inter-atrial septum IS and toward tricuspid valve plane TVP, rather than jetting across right atrium RA and dissecting or otherwise disrupting the natural vortical flow pattern. In this way, the flow of blood out of cardiovascular implant device 400 can join with blood flowing down along inter-atrial septum IS and merge into the right atrial vortex.

Cardiovascular implant device 400, including guide wall 450, can minimize disruption to or potentially enhance the natural flow patterns local to a site where cardiovascular implant device 400 is implanted in heart H. When cardiovascular implant device 400 is implanted in inter-atrial septum IS, blood flowing from left atrium LA to right atrium RA through cardiovascular implant device 400 can be less disruptive to the natural rotational (e.g., vortical) flow pattern in right atrium RA because—in contrast to traditional septal shunt devices which may cause blood flow to jet across the right atrium—the flow out of cardiovascular implant device 400 is aligned with the natural vortical flow pattern. Utilizing guide wall 450 to align the flow out of cardiovascular implant device 400 with the natural flow pattern in a chamber or vessel of heart H minimizes any disruptions to the natural flow pattern that could otherwise result from implantation of a traditional shunt device with no directional component. Further, aligning the flow out of cardiovascular implant device 400 could also potentially mitigate reduced flow due to a pathophysiology or other cause or enhance baseline flow. As a result, cardiovascular implant device 400 can maintain kinetic energy of the cardiovascular blood flow, which in turn reduces the cardiac work needed and improves cardiac efficiency. These hemodynamic effects can potentially improve patient outcomes after receiving cardiovascular implant device 400 because cardiovascular implant device 400 can be more effective and potentially safer.

### Device 500 (FIGS. 10A-10B)

FIG. 10A is a schematic cross-sectional view of heart H illustrating cardiovascular implant device 500 positioned in inter-atrial septum IS and including internal blades 560. FIG. 10B is an enlarged schematic cross-sectional view of inter-atrial septum IS illustrating details of cardiovascular implant device 500. FIGS. 10A and 10B will be discussed together. As illustrated in FIGS. 10A-10B, cardiovascular implant device 500 includes annular body 502, which includes struts 503, central flow tube 504, and flow path 506; and anchoring members 508. Central flow tube 504 includes inflow end 510, outflow end 512, and flow surface 514. Cardiovascular implant device 500 further includes blades 560 and shaft 562. Blades 560 each include root portion 564 and tip portion 566. FIGS. 10A-10B show right atrium RA, left atrium LA, and inter-atrial septum IS. FIG. 10A also shows heart H, superior vena cava SVC, inferior vena cava IVC, tricuspid valve TV, pulmonary veins PVS, and mitral valve MV. FIGS. 10A-10B further show tissue wall plane TWP, outer diameter OD, axis AX5, and angle α5. FIG. 10A also shows right atrial vortical flow RVF and tricuspid valve plane TVP.

Cardiovascular implant device 500 is an implantable device for use in a cardiovascular system. Cardiovascular implant device 500 is configured to be implanted in blood vessels or chambers of heart H. In the illustrated example, cardiovascular implant device 500 is a flow directing shunt device for shunting blood from one vessel or chamber to another vessel or chamber. Specifically, as shown in FIGS. 10A-10B, cardiovascular implant device 500 is positioned in inter-atrial septum IS. In other examples, cardiovascular implant device 500 can be positioned in any other tissue wall between adjacent chambers and/or vessels of heart H (or the cardiovascular system). Cardiovascular implant device 500 can be delivered into the cardiovascular system via a catheter (i.e., transcatheter delivery) or can be surgically placed using transcatheter or surgical procedures known in the art.

Annular body 502 is a main body portion of cardiovascular implant device 500.

Annular body 502 can be expandable. Annular body 502 is generally cylindrical and is tubular in cross-section but can have a wide variety of different shapes and sizes. Annular body 502 can press against or into tissue walls at the implant site or contour (or extend) around anatomical structures of the cardiovascular system to set and maintain the position of cardiovascular implant device 500. In some examples, e.g., as shown in FIGS. 10A-10B, annular body 502 can be formed of a plurality of struts 503. Struts 503 can make up a lattice or mesh of annular body 502 and define openings therein. In such examples, annular body 502 can be a stent frame structure for supporting a graft material that guides flow through cardiovascular implant device 500. In other examples, annular body 502 can be solidly formed.

Annular body 502 can be positioned in a puncture in a tissue wall to hold the tissue wall open around annular body 502 so that blood can flow between blood vessels or chambers of heart H through cardiovascular implant device 500. In the example shown in FIGS. 10A-10B, annular body 502 is positioned in a puncture in inter-atrial septum IS between left atrium LA and right atrium RA so that blood can flow from left atrium LA to right atrium RA through cardiovascular implant device 500. In some examples, struts 503 of annular body 502 form a cage of sorts that is sufficient to hold the tissue wall open around annular body 502. In other examples, a material from which annular body 502 is solidly formed is sufficient to hold the tissue wall open around annular body 502.

Annular body 502 has outer diameter OD. Outer diameter OD is the diameter of annular body 502 as measured to an exterior surface of cardiovascular implant device 500. Outer diameter OD is configured to be approximately the same size as a puncture diameter in a tissue wall in which cardiovascular implant device 500 will be implanted so annular body 502 is able to fit within the puncture. Outer diameter OD can have any size such that cardiovascular implant device 500 is dimensioned to be suitable for a variety of different patient conditions and/or anatomies. In some examples, outer diameter OD can also vary along a length of annular body 502 based on an overall shape or profile of annular body 502.

Annular body 502 includes central flow tube 504, which serves as a conduit for guiding flow through cardiovascular implant device 500. Central flow tube 504 surrounds flow path 506. Flow path 506 is an opening extending through central flow tube 504 such that cardiovascular implant device 500 is open at each opposing end. Flow path 506 is the path through which blood flows or is directed through cardiovascular implant device 500. Central flow tube 504 includes flow surface 514, which is configured to be a flow contacting surface when cardiovascular implant device 500 is implanted in a vessel or chamber of heart H. Flow surface 514 is a radially inner surface of central flow tube 504. Flow path 506 through central flow tube 504 is defined by flow surface 514.

A profile of central flow tube 504 and flow path 506 can be straight, curved, a combination of straight and curved sections, or any other suitable shape. In some examples, the profile of central flow tube 504 and flow path 506 can be defined by or the same as a profile of annular body 502 (e.g., as shown in FIG. 5). In other examples, the profile of central flow tube 504 and flow path 506 can be independent of or different from the profile of annular body 502 (e.g., as shown in FIG. 6). Similarly, a cross-sectional shape or profile of central flow tube 504 and annular body 502 can be the same, for example, circular, oval, etc. Alternatively, central flow tube 504 and annular body 502 can have different cross-sectional shapes. For example, annular body 502 could have a circular cross-section and central flow tube 504 could have an oval cross-section. Moreover, the cross-sectional shape of central flow tube 504 and/or annular body can also vary along the length of either. The cross-sectional shape of central flow tube 504 can be selected at various points along its length, such as at outflow end 512, to affect the flow direction.

Central flow tube 504 (and flow path 506 therein) extends from inflow end 510 and outflow end 512. Inflow end 510 can be an end of central flow tube 504 that is relatively upstream of outflow end 512 with respect to a flow of blood through cardiovascular implant device 500, as represented by arrow F in FIGS. 10A-10B, when cardiovascular implant device 500 is implanted in a blood vessel or chamber of heart H. Accordingly, outflow end 512 is an end of central flow tube 504 that is relatively downstream of inflow end 510 with respect to a flow of blood through cardiovascular implant device 500, as represented by arrow F in FIGS. 10A-10B, when cardiovascular implant device 500 is implanted in a blood vessel or chamber of heart H. In the example shown in FIGS. 10A-10B, inflow end 510 is positioned on a left atrial side of inter-atrial septum IS and outflow end 512 is positioned downstream on a right atrial side of inter-atrial septum IS so blood can flow from left atrium LA to right atrium RA through flow path 506. As illustrated in FIGS. 10A-10B, inflow end 510 can be essentially flush with the left atrial side of inter-atrial septum IS, whereas outflow end 512 can, in some examples, be spaced away from the right atrial side of inter-atrial septum IS within right atrium RA (i.e., cardiovascular implant device 500 can extend further into right atrium RA at outflow end 512 than into left atrium LA at inflow end 510). In other examples, either inflow end 510 or outflow end 512 or both can be flush with or spaced away from the respective side of a tissue wall. Although inflow end 510 is defined as being relatively upstream of outflow end 512, it should be understood that other actual positions of inflow end 510 or outflow end 512 are possible depending on the location where cardiovascular implant device 500 is implanted. Central flow tube 504 can have any suitable length as measured from inflow end 510 to outflow end 512. For example, central flow tube 504 can be designed to have a length that approximates the thickness of inter-atrial septum IS or another tissue wall in which cardiovascular implant device 500 is positioned. In other examples, central flow tube 504 can be longer or shorter than a thickness of inter-atrial septum IS or another tissue wall.

In general, central flow tube 504 can be formed of any suitable material for forming a tubular structure that surrounds flow path 506. For example, all or a portion of central flow tube 504 can be formed of a graft material. The graft material can be a synthetic material, such as a woven polyester or a polytetrafluoroethylene (PTFE), a biologic material, a metallic material, or other materials, to name a few non-limiting examples. Central flow tube 504 formed of a graft material can be supported in cardiovascular implant device 500 by struts 503 of annular body 502. In such examples, central flow tube 504 can be attached to struts 503 of annular body 502 by any suitable attachment means, such as by stitching, gluing, tying, etc. In other examples, central flow tube 504 can be solidly formed with annular body 504.

One or more anchoring members 508 extend outward from annular body 502. Anchoring members 508 hold cardiovascular implant device 500 in position in a tissue wall when cardiovascular implant device 500 is implanted in the body. Anchoring members 508 can take any suitable form for securing cardiovascular implant device 500 to a tissue wall. In some examples, anchoring members 508 can be one or more arms. In other examples, anchoring member 508 can be a flange or annular lip that is configured to have a larger diameter than a diameter of the puncture or opening in which cardiovascular implant device 500 is positioned such that cardiovascular implant device 500 is prevented from slipping through the puncture or opening. In some examples, anchoring members 508 can be curved toward the tissue wall or, alternatively, can rest flush against the tissue wall. As illustrated in FIGS. 10A-10B, cardiovascular implant device 500 can include one or more anchoring members 508 extending from one end of central flow tube 504. Specifically, cardiovascular implant device 500 can include anchoring members 508 adjacent inflow end 510. In other examples, cardiovascular implant device 500 can include anchoring members 508 adjacent outflow end 512. In yet other examples, cardiovascular implant device 500 can include anchoring members 508 at both inflow end 510 and outflow end 512.

As illustrated in FIGS. 10A-10B, cardiovascular implant device 500 includes blades 560 connected to shaft 562. Shaft 562 extends longitudinally through central flow tube 504. In some examples, shaft 562 is attached to central flow tube 504 by an extension of wire or other attachment mechanism at inflow end 510 and outflow end 512 or at other positions along the length of central flow tube 504. In general, the structures in the interior of central flow tube 504, including blades 560 and shaft 562, can be configured to be collapsible or can have physical dimensions sized to avoid interfering with other components if cardiovascular implant device 500 will be delivered with a catheter. Surgical delivery examples may not have the same size-related limitations for implementing blades 560.

Blades 560 extend radially about shaft 562. Each of blades 560 includes a respective root portion 564 and tip portion 566. Root portion 564 is a proximal portion of blade 560 adjacent shaft 562. Tip portion 566 is a distal portion of blade 560. Each of blades 560 extends radially from root portion 564 to tip portion 566 (or from shaft 562 toward flow surface 514). Tip portions 566 can be spaced away from flow surface 514.

Blades 560 can be arranged in one or more sets of blades 560 along the length of shaft 562. Blades 560 can also be arranged in a ring or rings around shaft 562. That is, although FIGS. 10A-10B shows one set of blades 560, other examples could include multiple sets of blades 560 arranged in separate rings. In one example, a set of blades 560 is a stator (i.e., blades 560 are stationary). In another example, a set of blades 560 is a rotor (i.e., blades 560 can rotate). In such examples, shaft 562 can include a concentric stationary shaft and rotatable portion to which blades 560 are connected. Stator, rotor, or a combination of stator and rotor sets of blades 560 can be selected based on desired flow characteristics of blood flowing through cardiovascular implant device 500.

Blades 560 are flow directing components of cardiovascular implant device 500. More specifically, blades 560 are arranged and positioned to guide the flow of blood through central flow tube 504 and to direct the flow of blood out of cardiovascular implant device 500 in a particular direction. As illustrated in FIG. 10A, blades 560 are configured to direct the flow of blood out of cardiovascular implant device 500 toward tricuspid valve plane TVP (a plane that includes the annulus of tricuspid valve TV). When cardiovascular implant device 500 is implanted in inter-atrial septum IS, blades 560 are positioned to align the flow of blood out of cardiovascular implant device 500 with the natural flow pattern in right atrium RA. As illustrated in FIGS. 10A-10B, axis AX5 is a longitudinal axis aligned with the flow of blood out of central flow tube 504. Axis AX5 forms angle α5 with tissue wall plane TWP of the tissue wall (e.g., inter-atrial septum IS) in which cardiovascular implant device 500 is configured to be positioned. Tissue wall plane TWP is a vertical reference plane defined by the tissue wall and so will be approximately perpendicular to flow path 506 where it crosses the tissue wall. Angle α5 can be considered an exit angle of the blood flowing out of cardiovascular implant device 400. In some examples, angle α5 is between zero and seventy-five degrees (0°-75°).

Once cardiovascular implant device 500 is implanted in cardiovascular system (e.g., inter-atrial septum IS as shown in FIGS. 10A-10B or another tissue wall), circulating blood passes through flow path 506 of cardiovascular implant device 500. In the example shown in FIGS. 10A-10B, blood flows from left atrium LA, through flow path 506, and into right atrium RA. As blood flows through cardiovascular implant device 500, blades 560 guide the blood flow so that the flow of blood out of cardiovascular implant device 500 aligns and joins with a natural flow pattern of blood in right atrium RA. Blades 560 can also impart rotational velocity to the flow of blood through flow path 506 to help align the flow. More specifically, blades 560 guide the flow of blood through cardiovascular implant device 500 to align and join the flow of blood out of cardiovascular implant device 500 with a natural vortical flow pattern of blood (i.e., the right-sided flow vortex) in right atrium RA (indicated in FIG. 10A by the schematic stream lines labeled RVF). As illustrated by arrow F in FIG. 10A, the flow of blood out of cardiovascular implant device 500 is directed in a curved path along the right atrial side of inter-atrial septum IS and toward tricuspid valve plane TVP, rather than jetting across right atrium RA and dissecting or otherwise disrupting the natural vortical flow pattern. In this way, the flow of blood out of cardiovascular implant device 500 can join with blood flowing down along inter-atrial septum IS and merge into the right atrial vortex.

Cardiovascular implant device 500, including blades 560, can minimize disruption to or potentially enhance the natural flow patterns local to a site where cardiovascular implant device 500 is implanted in heart H. When cardiovascular implant device 500 is implanted in inter-atrial septum IS, blood flowing from left atrium LA to right atrium RA through cardiovascular implant device 500 can be less disruptive to the natural rotational (e.g., vortical) flow pattern in right atrium RA because—in contrast to traditional septal shunt devices which may cause blood flow to jet across the right atrium—the flow out of cardiovascular implant device 500 is aligned with the natural vortical flow pattern. Utilizing blades 560 to align the flow out of cardiovascular implant device 500 with the natural flow pattern in a chamber or vessel of heart H minimizes any disruptions to the natural flow pattern that could otherwise result from implantation of a traditional shunt device with no directional component. Further, aligning the flow out of cardiovascular implant device 500 could also potentially mitigate reduced flow due to a pathophysiology or other cause or enhance baseline flow. As a result, cardiovascular implant device 500 can maintain kinetic energy of the cardiovascular blood flow, which in turn reduces the cardiac work needed and improves cardiac efficiency. These hemodynamic effects can potentially improve patient outcomes after receiving cardiovascular implant device 500 because cardiovascular implant device 500 can be more effective and potentially safer.

### Devices 600 and 100A (FIGS. 11-13)

FIG. 11 is a schematic cross-sectional view of heart H illustrating cardiovascular implant device 600 positioned in inter-atrial septum IS and including adjustable portion 670. FIG. 12A is an enlarged schematic view of adjustable portion 670 in compressed configuration 680. FIG. 12B is an enlarged schematic view of adjustable portion 670 in expanded configuration 685. FIGS. 11-12B will be discussed together. As illustrated in FIG. 11, cardiovascular implant device 600 includes annular body 602, which includes struts 603, central flow tube 604 and flow path 606; and anchoring members 608. Central flow tube 604 includes inflow end 610, outflow end 612, and flow surface 614. Central flow tube 604 further includes straight portion 620 and adjustable portion 670. Adjustable portion 670 includes accordion folds 672. FIG. 11 also shows heart H, right atrium RA, left atrium LA, superior vena cava SVC, inferior vena cava IVC, tricuspid valve TV, pulmonary veins PVS, mitral valve MV, inter-atrial septum IS. FIG. 11 further shows right atrial vortical flow RVF, tissue wall plane TWP, tricuspid valve plane TVP, outer diameter OD, axis AX6, and angle α6. FIG. 12A shows compressed configuration 680, and FIG. 12B shows expanded configuration 685.

Cardiovascular implant device 600 is an implantable device for use in a cardiovascular system. Cardiovascular implant device 600 is configured to be implanted in blood vessels or chambers of heart H. In the illustrated example, cardiovascular implant device 600 is a flow directing shunt device for shunting blood from one vessel or chamber to another vessel or chamber. Specifically, as shown in FIG. 11, cardiovascular implant device 600 is positioned in inter-atrial septum IS. In other examples, cardiovascular implant device 600 can be positioned in any other tissue wall between adjacent chambers and/or vessels of heart H (or the cardiovascular system). Cardiovascular implant device 600 can be delivered into the cardiovascular system via a catheter (i.e., transcatheter delivery) or can be surgically placed using transcatheter or surgical procedures known in the art.

Annular body 602 is a main body portion of cardiovascular implant device 600. Annular body 602 can be expandable. Annular body 602 is generally cylindrical and is tubular in cross-section but can have a wide variety of different shapes and sizes. Annular body 602 can press against or into tissue walls at the implant site or contour (or extend) around anatomical structures of the cardiovascular system to set and maintain the position of cardiovascular implant device 600. In some examples, e.g., as shown in FIG. 11, annular body 602 can be formed of a plurality of struts 603. Struts 603 can make up a lattice or mesh of annular body 602 and define openings therein. In such examples, annular body 602 can be a stent frame structure for supporting a graft material that guides flow through cardiovascular implant device 600. In other examples, annular body 602 can be solidly formed.

Annular body 602 can be positioned in a puncture in a tissue wall to hold the tissue wall open around annular body 602 so that blood can flow between blood vessels or chambers of heart H through cardiovascular implant device 600. In the example shown in FIG. 11, annular body 602 is positioned in a puncture in inter-atrial septum IS between left atrium LA and right atrium RA so that blood can flow from left atrium LA to right atrium RA through cardiovascular implant device 600. In some examples, struts 603 of annular body 602 form a cage of sorts that is sufficient to hold the tissue wall open around annular body 602. In other examples, a material from which annular body 602 is solidly formed is sufficient to hold the tissue wall open around annular body 602.

Annular body 602 has outer diameter OD. Outer diameter OD is the diameter of annular body 602 as measured to an exterior surface of cardiovascular implant device 600. Outer diameter OD is configured to be approximately the same size as a puncture diameter in a tissue wall in which cardiovascular implant device 600 will be implanted so annular body 602 is able to fit within the puncture. Outer diameter OD can have any size such that cardiovascular implant device 600 is dimensioned to be suitable for a variety of different patient conditions and/or anatomies. In some examples, outer diameter OD can also vary along a length of annular body 602 based on an overall shape or profile of annular body 602.

Annular body 602 includes central flow tube 604, which serves as a conduit for guiding flow through cardiovascular implant device 600. Central flow tube 604 surrounds flow path 606. Flow path 606 is an opening extending through central flow tube 604 such that cardiovascular implant device 600 is open at each opposing end. Flow path 606 is the path through which blood flows or is directed through cardiovascular implant device 600. Central flow tube 604 includes flow surface 614, which is configured to be a flow contacting surface when cardiovascular implant device 600 is implanted in a vessel or chamber of heart H. Flow surface 614 is a radially inner surface of central flow tube 604. Flow path 606 through central flow tube 604 is defined by flow surface 614.

A profile of central flow tube 604 and flow path 606 can be straight, curved, a combination of straight and curved sections, or any other suitable shape. In some examples, the profile of central flow tube 604 and flow path 606 can be defined by or the same as a profile of annular body 602 (e.g., as shown in FIG. 5). In other examples, the profile of central flow tube 604 and flow path 606 can be independent of or different from the profile of annular body 602 (e.g., as shown in FIG. 6). Similarly, a cross-sectional shape or profile of central flow tube 604 and annular body 602 can be the same, for example, circular, oval, etc. Alternatively, central flow tube 604 and annular body 602 can have different cross-sectional shapes. For example, annular body 602 could have a circular cross-section and central flow tube 604 could have an oval cross-section. Moreover, the cross-sectional shape of central flow tube 604 and/or annular body can also vary along the length of either. The cross-sectional shape of central flow tube 604 can be selected at various points along its length, such as at outflow end 612, to affect the flow direction.

Central flow tube 604 (and flow path 606 therein) extends from inflow end 610 and outflow end 612. Inflow end 610 can be an end of central flow tube 604 that is relatively upstream of outflow end 612 with respect to a flow of blood through cardiovascular implant device 600, as represented by arrow Fin FIG. 11, when cardiovascular implant device 600 is implanted in a blood vessel or chamber of heart H. Accordingly, outflow end 612 is an end of central flow tube 604 that is relatively downstream of inflow end 610 with respect to a flow of blood through cardiovascular implant device 600, as represented by arrow F in FIG. 11, when cardiovascular implant device 600 is implanted in a blood vessel or chamber of heart H. In the example shown in FIG. 11, inflow end 610 is positioned on a left atrial side of inter-atrial septum IS and outflow end 612 is positioned downstream on a right atrial side of inter-atrial septum IS so blood can flow from left atrium LA to right atrium RA through flow path 606. As illustrated in FIG. 11, inflow end 610 can be essentially flush with the left atrial side of inter-atrial septum IS, whereas outflow end 612 can, in some examples, be spaced away from the right atrial side of inter-atrial septum IS within right atrium RA (i.e., cardiovascular implant device 600 can extend further into right atrium RA at outflow end 612 than into left atrium LA at inflow end 610). In other examples, either inflow end 610 or outflow end 612 or both can be flush with or spaced away from the respective side of a tissue wall. Although inflow end 610 is defined as being relatively upstream of outflow end 612, it should be understood that other actual positions of inflow end 610 or outflow end 612 are possible depending on the location where cardiovascular implant device 600 is implanted. Central flow tube 604 can have any suitable length as measured from inflow end 610 to outflow end 612. For example, central flow tube 604 can be designed to have a length that approximates the thickness of inter-atrial septum IS or another tissue wall in which cardiovascular implant device 600 is positioned. In other examples, central flow tube 604 can be longer or shorter than a thickness of inter-atrial septum IS or another tissue wall.

In general, central flow tube 604 can be formed of any suitable material for forming a tubular structure that surrounds flow path 606. For example, all or a portion of central flow tube 604 can be formed of a graft material. The graft material can be a synthetic material, such as a woven polyester or a polytetrafluoroethylene (PTFE), a biologic material, a metallic material, or other materials, to name a few non-limiting examples. Central flow tube 604 formed of a graft material can be supported in cardiovascular implant device 600 by struts 603 of annular body 602. In such examples, central flow tube 604 can be attached to struts 603 of annular body 602 by any suitable attachment means, such as by stitching, gluing, tying, etc. In other examples, central flow tube 604 can be solidly formed with annular body 604.

One or more anchoring members 608 extend outward from annular body 602. Anchoring members 608 hold cardiovascular implant device 600 in position in a tissue wall when cardiovascular implant device 600 is implanted in the body. Anchoring members 608 can take any suitable form for securing cardiovascular implant device 600 to a tissue wall. In some examples, anchoring members 608 can be one or more arms. In other examples, anchoring member 608 can be a flange or annular lip that is configured to have a larger diameter than a diameter of the puncture or opening in which cardiovascular implant device 600 is positioned such that cardiovascular implant device 600 is prevented from slipping through the puncture or opening. In some examples, anchoring members 608 can be curved toward the tissue wall or, alternatively, can rest flush against the tissue wall. As illustrated in FIG. 11, cardiovascular implant device 600 can include one or more anchoring members 608 extending from one end of central flow tube 604. Specifically, cardiovascular implant device 600 can include anchoring members 608 adjacent inflow end 610. In other examples, cardiovascular implant device 600 can include anchoring members 608 adjacent outflow end 612. In yet other examples, cardiovascular implant device 600 can include anchoring members 608 at both inflow end 610 and outflow end 612.

As illustrated in FIG. 11, central flow tube 604 includes straight portion 620 and adjustable portion 670. Straight portion 620 is a first portion or segment of central flow tube 604. In the example shown in FIG. 11, straight portion 620 is adjacent to and extends from inflow end 610 to capture blood flowing into cardiovascular implant device 600. A length of straight portion 620 is sized to span a puncture in a tissue wall within which cardiovascular implant device 600 is configured to be positioned. Adjustable portion 670 is a second portion or segment of central flow tube 604. Adjustable portion 670 is a second portion or segment of central flow tube 604. Adjustable portion 670 is connected to straight portion 620. In the example, shown in FIG. 11, adjustable portion 670 is adjacent to and extends from outflow end 612 to straight portion 620. That is, adjustable portion 670 is a relatively downstream portion of central flow tube 604 and straight portion 620 is a relatively upstream portion of central flow tube 604 with respect to a direction of blood flow through cardiovascular implant device 600 when implanted in a tissue wall. Adjustable portion 670 can be continuous with straight portion 620. Adjustable portion 670 is illustrated in FIG. 11 as being longer than straight portion 620; however, it should be understood that adjustable portion 670 and straight portion 620 can have any relative lengths with respect to each other.

Adjustable portion 670 is a flexible portion of central flow tube 604. Adjustable portion 670 is adjustable between compressed configuration (or state) 680 and one or more expanded configurations (or states) 685. Adjustable portion 670 is like an accordion and includes one or more accordion folds 672, which permit expansion and contraction of adjustable portion 670. In compressed configuration 680, as shown in FIG. 12A, the surface of adjustable portion 670 is folded tightly together at accordion folds 672. In expanded configuration 685, as shown in FIG. 12B, accordion folds 672 of adjustable portion 670 are separated (i.e., unfolded or pulled apart) some amount, as illustrated by the bidirectional arrows in FIG. 12B. For example, as shown in FIG. 11, one side of accordion folds 672 can be separated more than the laterally opposite side of the same accordion folds 672 so that adjustable portion 670 is curved. Adjustable portion 670 has an essentially infinite number of expanded configurations 685, individual ones of which can be accomplished by varying the combination of which ones of accordion folds 672 are separated or compressed, which side of accordion folds 672 are separated or compressed, and how much ones of accordion folds 672 are separated or compressed. It should be understood that only one such expanded configuration 685 is shown in FIG. 12B, and many other expanded configurations 685 are possible. A particular implementation of adjustable portion 670 can be limited in how much it expands by adjusting the number, size, and/or spacing of accordion folds 672 in the design. The possible expanded states 685 of adjustable portion 670 can be selected based on a desired characteristic of the flow out of cardiovascular implant device 600. In some examples, adjustable portion 670 (or a portion of annular body 602 that provides support at adjustable portion 670) is formed of an elastic material to accommodate expansion and contraction of adjustable portion 670. For example, the elastic material can be a cobalt-chromium alloy.

Adjustable portion 670 is a flow directing component of cardiovascular implant device 600. Adjustable portion 670 is positioned or positionable to direct the flow of blood out of cardiovascular implant device 600 in a particular direction. More specifically, adjustable portion 670 is adjustable to direct the flow of blood out of cardiovascular implant device 600 in a particular direction. As illustrated in FIG. 11, adjustable portion 670 is configured to curve toward tricuspid valve plane TVP (a plane that includes the annulus of tricuspid valve TV) by the positioning of cardiovascular implant device 600 and the expansion of accordion folds 672 so that adjustable portion 670 is in one of its one or more expanded configurations 685. Accordingly, adjustable portion 670 is configured to direct the flow out of cardiovascular implant device 600 toward tricuspid valve plane TVP. In its one or more expanded configurations 685, adjustable portion 670 defines a turn in flow path 606. When cardiovascular implant device 600 is implanted in inter-atrial septum IS, the turn aligns the portion of flow path 606 at outflow end 612 with the natural flow pattern in right atrium RA. Axis AX6 drawn longitudinally through outflow end 612 (which approximates a longitudinal axis aligned with the flow of blood out of central flow tube 604) forms angle α6 with tissue wall plane TWP of the tissue wall (e.g., inter-atrial septum IS) in which cardiovascular implant device 600 is configured to be positioned. Tissue wall plane TWP is a vertical reference plane defined by the tissue wall and so will be approximately perpendicular to flow path 606 where it crosses the tissue wall. In some examples, angle α6 is between zero and seventy-five degrees (0°-75°).

Because adjustable portion 670 is adjacent outflow end 612, adjustable portion 670 is configured to face or extend partially into right atrium RA when cardiovascular implant device 600 is implanted in inter-atrial septum IS. The projection of adjustable portion 670 into right atrium RA can be minimized so that adjustable portion 670 only projects into right atrium RA sufficient to fix cardiovascular implant device 600 in place in inter-atrial septum IS. In some examples, adjustable portion 670 is biased into expanded configuration 685 (e.g., the configuration illustrated in FIGS. 11 and 12B). In other examples, adjustable portion 670 can be configured to self-expand or self-orient to some degree based on the pressure difference between left atrium LA and right atrium RA. In such examples, when there is a greater pressure difference between left atrium LA and right atrium RA, flow through cardiovascular implant device 600 can force adjustable portion 670 to expand more to change the orientation of outflow end 612. Adjustable portion 670 can be configured such that angle α6 is an angle that corresponds to a maximum expanded state of adjustable portion 670. The orientation of adjustable portion 670 in response to increased flow through cardiovascular implant device 600 can be selected based on desired characteristics of the flow out of cardiovascular implant device 600.

Once cardiovascular implant device 600 is implanted in cardiovascular system (e.g., inter-atrial septum IS as shown in FIG. 11 or another tissue wall), circulating blood passes through flow path 606 of cardiovascular implant device 600. In the example shown in FIG. 11, blood flows from left atrium LA, through flow path 606, and into right atrium RA. As blood flows out of cardiovascular implant device 600, adjustable portion 670 aligns the flow of blood with a natural flow pattern of blood in right atrium RA so that the flow of blood out of cardiovascular implant device 600 joins with the natural flow pattern of blood in right atrium RA. More specifically, adjustable portion 670 aligns the flow of blood out of cardiovascular implant device 600 with a natural vortical flow pattern of blood (i.e., the right-sided flow vortex) in right atrium RA (indicated in FIG. 11 by the schematic stream lines labeled RVF). As illustrated by arrow F in FIG. 11, the flow of blood out of cardiovascular implant device 600 is directed in a curved path along the right atrial side of inter-atrial septum IS and toward tricuspid valve plane TVP, rather than jetting across right atrium RA and dissecting or otherwise disrupting the natural vortical flow pattern. In this way, the flow of blood out of cardiovascular implant device 600 can join with blood flowing down along inter-atrial septum IS and merge into the right atrial vortex.

Additionally, adjustable portion 670 is configured to be adjusted into one of the one or more expanded configurations 685 shortly prior to or during an implantation procedure for cardiovascular implant device 600. Accordion folds 672 can be unfolded or compressed to adjust the curvature of adjustable portion 670 based on patient requirements, such as particular anatomical or flow conditions. In this way, adjustable portion 670 can have curvature that is configured specific to a patient in which cardiovascular implant device 600 will be or is implanted. In examples where adjustable portion 670 is adjusted during an implantation procedure, the orientation of adjustable portion 670 can be visualized in real time. For example, the orientation of adjustable portion 670 can be visualized with fluoroscopy using radiopaque markers or contrast dye or with other visualization techniques known in the art. Moreover, a delivery device for cardiovascular implant device 600 can be modified in such examples to include a guidewire and snare or similar mechanism that can releasably attach to adjustable portion 670 for translating force from the physician's movement into folding or unfolding accordion folds 672 to adjust the orientation of adjustable portion 670 (i.e., to select the desired expanded configuration 685). Accordingly, cardiovascular implant device 600 can be delivered with adjustable portion 670 in one configuration (e.g., an initial expanded configuration 685), and a physician can further adjust the configuration of adjustable portion 670 based on observations about the patient's anatomy or flow conditions.

Cardiovascular implant device 600, including adjustable portion 670, can minimize disruption to or potentially enhance the natural flow patterns local to a site where cardiovascular implant device 600 is implanted in heart H. When cardiovascular implant device 600 is implanted in inter-atrial septum IS, blood flowing from left atrium LA to right atrium RA through cardiovascular implant device 600 can be less disruptive to the natural rotational (e.g., vortical) flow pattern in right atrium RA because—in contrast to traditional septal shunt devices which may cause blood flow to jet across the right atrium—the flow out of cardiovascular implant device 600 is aligned with the natural vortical flow pattern. Utilizing adjustable portion 670 to align the flow out of cardiovascular implant device 600 with the natural flow pattern in a chamber or vessel of heart H minimizes any disruptions to the natural flow pattern that could otherwise result from implantation of a traditional shunt device with no directional component. Further, aligning the flow out of cardiovascular implant device 600 could also potentially mitigate reduced flow due to a pathophysiology or other cause or enhance baseline flow. As a result, cardiovascular implant device 600 can maintain kinetic energy of the cardiovascular blood flow, which in turn reduces the cardiac work needed and improves cardiac efficiency. These hemodynamic effects can potentially improve patient outcomes after receiving cardiovascular implant device 600 because cardiovascular implant device 600 can be more effective and potentially safer.

Further, adjustable portion 670 allows cardiovascular implant device 600 to be readily adjustable for a wider range of patient anatomies and conditions. That is, a particular expanded configuration 685 of adjustable portion 670 can be selected to best fit a particular patient's anatomy or flow condition, such as a right atrial flow pattern. Moreover, the configuration of adjustable portion 670 can be determined immediately before or during an implantation procedure, which allows for real-time adjustments to be made to the device based on information obtained by the physician about the patient.

FIG. 13 is a schematic cross-sectional view of inter-atrial septum IS illustrating cardiovascular implant device 600A positioned in inter-atrial septum IS and including varying inner diameter ID. As illustrated in FIG. 13, cardiovascular implant device 600A includes annular body 602A, which includes struts 603A, central flow tube 604A and flow path 606A; and anchoring members 608A. Central flow tube 604A includes inflow end 610A, outflow end 612A, and flow surface 614A. Central flow tube 604A further includes straight portion 620A and adjustable portion 670A. Adjustable portion 670A includes accordion folds 672A. FIG. 13 also shows right atrium RA, left atrium LA, and inter-atrial septum IS. FIG. 13 further shows tissue wall plane TWP, outer diameter OD, inner diameter ID, axis AX6, and angle α6.

Cardiovascular implant device 600A has a generally similar structure, design, and function to cardiovascular implant device 600 described above with reference to FIG. 11, except cardiovascular implant device 600A includes varying inner diameter ID.

Central flow tube 604A has inner diameter ID. Inner diameter ID is the diameter of central flow tube 604A as measured to flow surface 614A. In general, inner diameter ID can have any size such that central flow tube 604A and flow path 606A therethrough are dimensioned to accommodate the flow of blood through cardiovascular implant device 600A. In the example shown in FIG. 13, inner diameter ID varies along the length of central flow tube 604A. Specifically, central flow tube 604A is tapered such that inner diameter ID is narrower toward inflow end 610A and wider toward outflow end 612A. This funnel shape can help direct the flow of blood in a desired direction out of cardiovascular implant device 600A. In other examples, inner diameter ID can vary in different ways along the length of central flow tube 604A, such as tapering in the opposite direction, tapering along only a portion of central flow tube 604A, etc.

### Device 700 (FIG. 14)

FIG. 14 is a schematic cross-sectional view of heart H illustrating cardiovascular implant device 700 positioned in inter-atrial septum IS and including angled central flow tube 704. As illustrated in FIG. 14, cardiovascular implant device 700 includes annular body 702, which includes struts 703, central flow tube 704, and flow path 706; and anchoring members 708. Central flow tube 704 includes inflow end 710, outflow end 712, and flow surface 714. FIG. 14 also shows heart H, right atrium RA, left atrium LA, superior vena cava SVC, inferior vena cava IVC, tricuspid valve TV, pulmonary veins PVS, mitral valve MV, inter-atrial septum IS. FIG. 14 further shows right atrial vortical flow RVF, tissue wall plane TWP, tricuspid valve plane TVP, outer diameter OD, axis AX7, and angle α7.

Cardiovascular implant device 700 is an implantable device for use in a cardiovascular system. Cardiovascular implant device 700 is configured to be implanted in blood vessels or chambers of heart H. In the illustrated example, cardiovascular implant device 700 is a flow directing shunt device for shunting blood from one vessel or chamber to another vessel or chamber. Specifically, as shown in FIG. 14, cardiovascular implant device 700 is positioned in inter-atrial septum IS. In other examples, cardiovascular implant device 700 can be positioned in any other tissue wall between adjacent chambers and/or vessels of heart H (or the cardiovascular system). Cardiovascular implant device 700 can be delivered into the cardiovascular system via a catheter (i.e., transcatheter delivery) or can be surgically placed using transcatheter or surgical procedures known in the art.

Annular body 702 is a main body portion of cardiovascular implant device 700. Annular body 702 can be expandable. Annular body 702 is generally cylindrical and is tubular in cross-section but can have a wide variety of different shapes and sizes. Annular body 702 can press against or into tissue walls at the implant site or contour (or extend) around anatomical structures of the cardiovascular system to set and maintain the position of cardiovascular implant device 700. In some examples, e.g., as shown in FIG. 14, annular body 702 can be formed of a plurality of struts 703. Struts 703 can make up a lattice or mesh of annular body 702 and define openings therein. In such examples, annular body 702 can be a stent frame structure for supporting a graft material that guides flow through cardiovascular implant device 700. In other examples, annular body 702 can be solidly formed.

Annular body 702 can be positioned in a puncture in a tissue wall to hold the tissue wall open around annular body 702 so that blood can flow between blood vessels or chambers of heart H through cardiovascular implant device 700. In the example shown in FIG. 14, annular body 702 is positioned in a puncture in inter-atrial septum IS between left atrium LA and right atrium RA so that blood can flow from left atrium LA to right atrium RA through cardiovascular implant device 700. In some examples, struts 703 of annular body 702 form a cage of sorts that is sufficient to hold the tissue wall open around annular body 702. In other examples, a material from which annular body 702 is solidly formed is sufficient to hold the tissue wall open around annular body 702.

Annular body 702 has outer diameter OD. Outer diameter OD is the diameter of annular body 702 as measured to an exterior surface of cardiovascular implant device 700. Outer diameter OD is configured to be approximately the same size as a puncture diameter in a tissue wall in which cardiovascular implant device 700 will be implanted so annular body 702 is able to fit within the puncture. Outer diameter OD can have any size such that cardiovascular implant device 700 is dimensioned to be suitable for a variety of different patient conditions and/or anatomies. In some examples, outer diameter OD can also vary along a length of annular body 702 based on an overall shape or profile of annular body 702.

Annular body 702 includes central flow tube 704, which serves as a conduit for guiding flow through cardiovascular implant device 700. Central flow tube 704 surrounds flow path 706. Flow path 706 is an opening extending through central flow tube 704 such that cardiovascular implant device 700 is open at each opposing end. Flow path 706 is the path through which blood flows or is directed through cardiovascular implant device 700. Central flow tube 704 includes flow surface 714, which is configured to be a flow contacting surface when cardiovascular implant device 700 is implanted in a vessel or chamber of heart H. Flow surface 714 is a radially inner surface of central flow tube 704. Flow path 706 through central flow tube 704 is defined by flow surface 714.

A profile of central flow tube 704 and flow path 706 can be straight, curved, a combination of straight and curved sections, or any other suitable shape. In some examples, the profile of central flow tube 704 and flow path 706 can be defined by or the same as a profile of annular body 702 (e.g., as shown in FIG. 5). In other examples, the profile of central flow tube 704 and flow path 706 can be independent of or different from the profile of annular body 702 (e.g., as shown in FIG. 6). Similarly, a cross-sectional shape or profile of central flow tube 704 and annular body 702 can be the same, for example, circular, oval, etc. Alternatively, central flow tube 704 and annular body 702 can have different cross-sectional shapes. For example, annular body 702 could have a circular cross-section and central flow tube 704 could have an oval cross-section. Moreover, the cross-sectional shape of central flow tube 704 and/or annular body 702 can also vary along the length of either. The cross-sectional shape of central flow tube 704 can be selected at various points along its length, such as at outflow end 712, to affect the flow direction.

Central flow tube 704 (and flow path 706 therein) extends from inflow end 710 and outflow end 712. Inflow end 710 can be an end of central flow tube 704 that is relatively upstream of outflow end 712 with respect to a flow of blood through cardiovascular implant device 700, as represented by arrow Fin FIG. 14, when cardiovascular implant device 700 is implanted in a blood vessel or chamber of heart H. Accordingly, outflow end 712 is an end of central flow tube 704 that is relatively downstream of inflow end 710 with respect to a flow of blood through cardiovascular implant device 700, as represented by arrow F in FIG. 14, when cardiovascular implant device 700 is implanted in a blood vessel or chamber of heart H. In the example shown in FIG. 14, inflow end 710 is positioned on a left atrial side of inter-atrial septum IS and outflow end 712 is positioned downstream on a right atrial side of inter-atrial septum IS so blood can flow from left atrium LA to right atrium RA through flow path 706. As illustrated in FIG. 14, inflow end 710 can be essentially flush with the left atrial side of inter-atrial septum IS, whereas outflow end 712 can, in some examples, be spaced away from the right atrial side of inter-atrial septum IS within right atrium RA (i.e., cardiovascular implant device 700 can extend further into right atrium RA at outflow end 712 than into left atrium LA at inflow end 710). In other examples, either inflow end 710 or outflow end 712 or both can be flush with or spaced away from the respective side of a tissue wall. Although inflow end 710 is defined as being relatively upstream of outflow end 712, it should be understood that other actual positions of inflow end 710 or outflow end 712 are possible depending on the location where cardiovascular implant device 700 is implanted. Central flow tube 704 can have any suitable length as measured from inflow end 710 to outflow end 712. For example, central flow tube 704 can be designed to have a length that approximates the thickness of inter-atrial septum IS or another tissue wall in which cardiovascular implant device 700 is positioned. In other examples, central flow tube 704 can be longer or shorter than a thickness of inter-atrial septum IS or another tissue wall.

In general, central flow tube 704 can be formed of any suitable material for forming a tubular structure that surrounds flow path 706. For example, all or a portion of central flow tube 704 can be formed of a graft material. The graft material can be a synthetic material, such as a woven polyester or a polytetrafluoroethylene (PTFE), a biologic material, a metallic material, or other materials, to name a few non-limiting examples. Central flow tube 704 formed of a graft material can be supported in cardiovascular implant device 700 by struts 703 of annular body 702. In such examples, central flow tube 704 can be attached to struts 703 of annular body 702 by any suitable attachment means, such as by stitching, gluing, tying, etc. In other examples, central flow tube 704 can be solidly formed with annular body 704.

One or more anchoring members 708 extend outward from annular body 702. Anchoring members 708 hold cardiovascular implant device 700 in position in a tissue wall when cardiovascular implant device 700 is implanted in the body. Anchoring members 708 can take any suitable form for securing cardiovascular implant device 700 to a tissue wall. In some examples, anchoring members 708 can be one or more arms. In other examples, anchoring member 708 can be a flange or annular lip that is configured to have a larger diameter than a diameter of the puncture or opening in which cardiovascular implant device 700 is positioned such that cardiovascular implant device 700 is prevented from slipping through the puncture or opening. In some examples, anchoring members 708 can be curved toward the tissue wall or, alternatively, can rest flush against the tissue wall. Cardiovascular implant device 700 can include one or more anchoring members 708 extending from one or both ends of central flow tube 704. In some examples, cardiovascular implant device 700 can include anchoring members 708 adjacent inflow end 710. In other examples, cardiovascular implant device 700 can include anchoring members 708 adjacent outflow end 712. In the example shown in FIG. 14, cardiovascular implant device 700 includes anchoring members 708 at both inflow end 710 and outflow end 712.

As illustrated in FIG. 14, central flow tube 704 is situated in cardiovascular implant device 700 such that it is configured to be angled with respect to tissue wall plane TWP. Tissue wall plane TWP is a vertical reference plane defined by the tissue wall (e.g., inter-atrial septum IS) in which cardiovascular implant device 700 is configured to be positioned. Thus, rather than being oriented approximately perpendicular to tissue wall plane TWP (e.g., as illustrated by the examples of devices shown in FIGS. 5-13), central flow tube 704 is configured to cross the tissue wall at a different angle. Flow path 706 through central flow tube 704 is also angled in the same manner as central flow tube 704 with respect to the tissue wall and tissue wall plane TWP. Accordingly, central flow tube 704 will also be referred to herein as “angled central flow tube 704,” and flow path 706 will also be referred to herein as “angled flow path 706”. Axis AX7 is longitudinal axis through central flow tube 704. Axis AX7 forms angle α7 with tissue wall plane TWP. In some examples, angle α7 is between zero and seventy-five degree (0°-75°). More generally, angle α7 can be less than about ninety degrees (<90°), whereas a perpendicular central flow tube would be at ninety degrees.

Angled central flow tube 704 is a flow directing component of cardiovascular implant device 700. Angled central flow tube 704 is positioned to direct the flow of blood out of cardiovascular implant device 700 in a particular direction. More specifically, angled central flow tube 704 is angled to direct the flow of blood out of cardiovascular implant device 700 in a particular direction. As illustrated in FIG. 14, angled central flow tube 704 (and angled flow path 706 therein) is configured by the positioning of cardiovascular implant device 700 to be angled toward tricuspid valve plane TVP (a plane that includes the annulus of tricuspid valve TV). Accordingly, angled central flow tube 704 is configured to direct the flow out of cardiovascular implant device 700 toward tricuspid valve plane TVP. When cardiovascular implant device 700 is implanted in inter-atrial septum IS, angled central flow tube 704 aligns the flow of blood through and out of cardiovascular implant device 700 with the natural flow pattern in right atrium RA. Axis AX7 approximates a longitudinal axis aligned with the flow of blood through and out of angled central flow tube 704. As described above, axis AX7 forms angle α7 with tissue wall plane TWP, so the flow of blood through and out of angled central flow tube 704 can also be described as forming angle α7 with tissue wall plane TWP. In some examples, angle α7 is between zero and seventy-five degrees (0°-75°).

Once cardiovascular implant device 700 is implanted in cardiovascular system (e.g., inter-atrial septum IS as shown in FIG. 14 or another tissue wall), circulating blood passes through flow path 706 of cardiovascular implant device 700. In the example shown in FIG. 14, blood flows from left atrium LA, through flow path 706, and into right atrium RA. As blood flows out of cardiovascular implant device 700, angled central flow tube 704 aligns the flow of blood with a natural flow pattern of blood in right atrium RA so that the flow of blood out of cardiovascular implant device 700 joins with the natural flow pattern of blood in right atrium RA. More specifically, angled central flow tube 704 aligns the flow of blood out of cardiovascular implant device 700 with a natural vortical flow pattern of blood (i.e., the right-sided flow vortex) in right atrium RA (indicated in FIG. 14 by the schematic stream lines labeled RVF). As illustrated by arrow F in FIG. 14, the flow of blood out of cardiovascular implant device 700 is directed in an angled path along the right atrial side of inter-atrial septum IS and toward tricuspid valve plane TVP, rather than jetting across right atrium RA and dissecting or otherwise disrupting the natural vortical flow pattern. In this way, the flow of blood out of cardiovascular implant device 700 can join with blood flowing down along inter-atrial septum IS and merge into the right atrial vortex.

Cardiovascular implant device 700, including angled central flow tube 704, can minimize disruption to or potentially enhance the natural flow patterns local to a site where cardiovascular implant device 700 is implanted in heart H. When cardiovascular implant device 700 is implanted in inter-atrial septum IS, blood flowing from left atrium LA to right atrium RA through cardiovascular implant device 700 can be less disruptive to the natural rotational (e.g., vortical) flow pattern in right atrium RA because—in contrast to traditional septal shunt devices which may cause blood flow to jet across the right atrium—the flow out of cardiovascular implant device 700 is aligned with the natural vortical flow pattern. Utilizing angled central flow tube 704 to align the flow out of cardiovascular implant device 700 with the natural flow pattern in a chamber or vessel of heart H minimizes any disruptions to the natural flow pattern that could otherwise result from implantation of a traditional shunt device with no directional component. Further, aligning the flow out of cardiovascular implant device 700 could also potentially mitigate reduced flow due to a pathophysiology or other cause or enhance baseline flow. As a result, cardiovascular implant device 700 can maintain kinetic energy of the cardiovascular blood flow, which in turn reduces the cardiac work needed and improves cardiac efficiency. These hemodynamic effects can potentially improve patient outcomes after receiving cardiovascular implant device 700 because cardiovascular implant device 700 can be more effective and potentially safer.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Examples

The following are non-exclusive descriptions of possible examples of the present invention.

A cardiovascular implant device includes an annular body, one or more anchoring members, and a flow directing component. The annular body includes a central flow tube extending from an inflow end to an outflow end and a flow path extending through the central flow tube. The one or more anchoring members extending outward from the annular body and are configured to secure the cardiovascular implant device to a tissue wall. The flow directing component is positioned to align a flow of blood out of the cardiovascular implant device with a natural flow pattern of blood in a right atrium so that the flow of blood out of the cardiovascular implant device joins with the natural flow pattern of blood in the right atrium.

The cardiovascular implant device of the preceding paragraph can optionally include, additionally and/or alternatively, any one or more of the following features, configurations, and/or additional components:

The flow directing component can be configured to direct the flow of blood out of the cardiovascular implant device toward a tricuspid valve plane in the right atrium.

The flow directing component can be positioned such that a longitudinal axis aligned with the flow of blood out of the cardiovascular implant device forms an angle of about zero to seventy-five degrees (0°-75°) with a vertical reference plane defined by the tissue wall.

The flow directing component can be a curved portion of the central flow tube; the curved portion can be adjacent the outflow end; and the curved portion can be curved to align the flow of blood out of the cardiovascular implant device with the natural flow pattern of blood in the right atrium so that the flow of blood out of the cardiovascular implant device joins with the natural flow pattern of blood in the right atrium.

The flow directing component can be a flap connected to the annular body at the outflow end of the central flow tube; and the flap can be angled to align the flow of blood out of the cardiovascular implant device with the natural flow pattern of blood in the right atrium so that the flow of blood out of the cardiovascular implant device joins with the natural flow pattern of blood in a right atrium.

The flow directing component can be a guide wall connected to a radially inner surface of the central flow tube; the flow path can be defined by the guide wall; and the guide wall can be positioned to guide a flow of blood through the central flow tube of the cardiovascular implant device so that the flow of blood out of the cardiovascular implant device aligns and joins with the natural flow pattern of blood in the right atrium.

The flow directing component can be a set of blades that extend radially about a shaft that extends longitudinally through the central flow tube; and the blades can be positioned to guide a flow of blood through the central flow tube of the cardiovascular implant device so that the flow of blood out of the cardiovascular implant device aligns and joins with the natural flow pattern of blood in the right atrium.

The flow directing component can be an adjustable portion of the central flow tube; the adjustable portion can be adjacent the outflow end; the adjustable portion can be adjustable between one or more expanded configurations and a compressed configuration; and the adjustable portion can be adjustable to align the flow of blood out of the cardiovascular implant device with the natural flow pattern of blood in the right atrium so that the flow of blood out of the cardiovascular implant device joins with the natural flow pattern of blood in the right atrium.

The flow directing component can be the central flow tube; and the central flow tube can be angled to align the flow of blood out of the cardiovascular implant device with the natural flow pattern of blood in the right atrium so that the flow of blood out of the cardiovascular implant device joins with the natural flow pattern of blood in the right atrium.

At least a portion of the central flow tube can be formed by a graft material.

The cardiovascular implant device can be sterilized.

At least a portion of the cardiovascular implant device can be formed of a shape-memory material.

A cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The central flow tube includes a curved portion adjacent the outflow end, the curved portion being curved to align a flow of blood out of the cardiovascular implant device with a natural flow pattern of blood in a right atrium so that the flow of blood out of the cardiovascular implant device joins with the natural flow pattern of blood in the right atrium.

The cardiovascular implant device of the preceding paragraph can optionally include, additionally and/or alternatively, any one or more of the following features, configurations, and/or additional components:

The central flow tube can further include a straight portion adjacent the inflow end, and the curved portion can be connected to the straight portion.

The straight portion can be sized to span a puncture in the tissue wall within which the cardiovascular implant device is configured to be positioned.

The curved portion can be configured to face the right atrium when the cardiovascular implant device is positioned in an inter-atrial septum.

The curved portion can be configured to curve toward a tricuspid valve plane in the right atrium.

A longitudinal axis through the outflow end can form an angle of about zero to seventy-five degrees (0°-75°) with a vertical reference plane defined by the tissue wall.

The curved portion can define a turn in the flow path.

The central flow tube, including the curved portion, can be formed by a graft material.

The cardiovascular implant device can be sterilized.

At least a portion of the cardiovascular implant device can be formed of a shape-memory material.

A cardiovascular implant device includes an annular body, one or more anchoring members, and a flap. The annular body includes a central flow tube extending from an inflow end to an outflow end and a flow path extending through the central flow tube. The one or more anchoring members extend outward from the annular body and are configured to secure the cardiovascular implant device to a tissue wall. The flap is connected to the annular body at the outflow end of the central flow tube. The flap is angled to align a flow of blood out of the cardiovascular implant device with a natural flow pattern of blood in a right atrium so that the flow of blood out of the cardiovascular implant device joins with the natural flow pattern of blood in the right atrium.

The cardiovascular implant device of the preceding paragraph can optionally include, additionally and/or alternatively, any one or more of the following features, configurations, and/or additional components:

The flap can be connected to the annular body with a flexible joint, and the flap can be positionable with respect to the annular body at the flexible joint.

The cardiovascular implant device can further include a stopper adjacent the flexible joint for preventing the flap from moving beyond a maximum opening angle.

The flap can be positioned such that it is angled toward a longitudinal axis through the central flow tube.

The flap can be solidly formed of a flexible material, or the flap can be formed of a wire frame and a cloth stretched over the wire frame.

The flap can be configured to extend into the right atrium.

The flap can be configured to direct the flow of blood out of the cardiovascular implant device toward a tricuspid valve plane in the right atrium.

The flap can be configured to form an angle of about zero to seventy-five degrees (0°-75°) with a vertical reference plane defined by the tissue wall.

The cardiovascular implant device can be sterilized.

At least a portion of the cardiovascular implant device can be formed of a shape-memory material.

A cardiovascular implant device is configured to be attached adjacent to an opening in a tissue wall between a right atrium and a left atrium of a heart. The cardiovascular implant device includes an anchoring member configured to secure the cardiovascular implant device to the tissue wall, a flexible joint connected to the anchoring member, and a flap connected to the flexible joint. The flap is angled to align a flow of blood out of the opening with a natural flow pattern of blood in the right atrium so that the flow of blood out of the puncture joins with the natural flow pattern of blood in the right atrium.

The cardiovascular implant device of the preceding paragraph can optionally include, additionally and/or alternatively, any one or more of the following features, configurations, and/or additional components:

The flap can be positionable with respect to the opening at the flexible joint.

The cardiovascular implant device can further include a stopper adjacent the flexible joint for preventing the flap from moving beyond a maximum opening angle.

The flap can be positioned such that it is angled toward a longitudinal axis through the opening.

The flap can be solidly formed of a flexible material, or the flap can be formed of a wire frame and a cloth stretched over the wire frame.

The flap can be configured to extend into the right atrium.

The flap can be configured to direct the flow of blood out of the opening toward a tricuspid valve plane in the right atrium.

The flap can be configured to form an angle of about zero to seventy-five degrees (0°-75°) with a vertical reference plane defined by the tissue wall.

The cardiovascular implant device can be sterilized.

At least a portion of the cardiovascular implant device can be formed of a shape-memory material.

A cardiovascular implant device includes an annular body and one or more anchoring members. The annular body includes a central flow tube extending from an inflow end to an outflow end, which includes a guide wall connected to a radially inner surface of the central flow tube, and a flow path extending through the central flow tube and defined by the guide wall. The one or more anchoring members extend outward from the annular body and are configured to secure the cardiovascular implant device to a tissue wall. The guide wall is positioned to guide a flow of blood through the central flow tube of the cardiovascular implant device so that a flow of blood out of the cardiovascular implant device aligns and joins with a natural flow pattern of blood in a right atrium.

The cardiovascular implant device of the preceding paragraph can optionally include, additionally and/or alternatively, any one or more of the following features, configurations, and/or additional components:

The guide wall can be attached circumferentially to the radially inner surface of the central flow tube.

The guide wall can be a spiral wall such that the flow path is a spiral path.

The spiral wall can extend from the inflow end to the outflow end within an interior of the central flow tube.

The guide wall can be positioned to prevent the flow of blood through the central flow tube from flowing in a straight path.

The guide wall can impart rotational velocity to the flow of blood through the central flow tube.

The guide wall can be configured to direct the flow of blood out of the cardiovascular implant device toward a tricuspid valve plane in the right atrium.

The guide wall can be positioned such that a longitudinal axis aligned with the flow of blood out of the cardiovascular implant device forms an angle of about zero to seventy-five degrees (0°-75°) with a vertical reference plane defined by the tissue wall.

The cardiovascular implant device can be sterilized.

At least a portion of the cardiovascular implant device can be formed of a shape-memory material.

A cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The cardiovascular implant device further includes a shaft extending longitudinally through the central flow tube and a set of blades extending radially about the shaft. The blades are positioned to guide a flow of blood through the central flow tube of the cardiovascular implant device so that a flow of blood out of the cardiovascular implant device aligns and joins with a natural flow pattern of blood in a right atrium.

The cardiovascular implant device of the preceding paragraph can optionally include, additionally and/or alternatively, any one or more of the following features, configurations, and/or additional components:

The blades can be arranged in a ring around the shaft.

The set of blades can be a stator.

The set of blades can be a rotor.

Each of the blades can include a root portion adjacent the shaft and a tip portion distal to the root portion such that the blade extends radially from the shaft toward an inner surface of the central flow tube, the tip portion being spaced from the inner surface of the central flow tube.

The blades can impart rotational velocity to the flow of blood through the central flow tube.

The blades can be configured to direct the flow of blood out of the cardiovascular implant device toward a tricuspid valve plane in the right atrium.

The blades can be arranged such that a longitudinal axis aligned with the flow of blood out of the cardiovascular implant device forms an angle of about zero to seventy-five degrees (0°-75°) with a vertical reference plane defined by the tissue wall.

The cardiovascular implant device can be sterilized.

At least a portion of the cardiovascular implant device can be formed of a shape-memory material.

A cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The central flow tube includes an adjustable portion adjacent the outflow end. The adjustable portion is adjustable between one or more expanded configurations and a compressed configuration and is adjustable to align a flow of blood out of the cardiovascular implant device with a natural flow pattern of blood in a right atrium so that the flow of blood out of the cardiovascular implant device joins with the natural flow pattern of blood in the right atrium.

The cardiovascular implant device of the preceding paragraph can optionally include, additionally and/or alternatively, any one or more of the following features, configurations, and/or additional components:

The adjustable portion can include one or more accordion folds.

The central flow tube can include an inner diameter, and the inner diameter can vary along a length of the central flow tube.

The central flow tube can further include a straight portion adjacent the inflow end, and the adjustable portion can be connected to the straight portion.

The straight portion can be sized to span a puncture in the tissue wall within which the cardiovascular implant device is configured to be positioned.

The adjustable portion can be configured to be adjusted into one of the one or more expanded configurations prior to an implantation procedure for the cardiovascular implant device.

The adjustable portion can be configured to be adjusted into one of the one or more expanded configurations during an implantation procedure for the cardiovascular implant device.

The adjustable portion can be configured to face the right atrium when the cardiovascular implant device is positioned in an inter-atrial septum.

The adjustable portion can be configured to curve toward a tricuspid valve plane in the right atrium when the adjustable portion is in one of the one or more expanded configurations.

A longitudinal axis through the outflow end can form an angle of about zero to seventy-five degrees (0°-75°) with a vertical reference plane defined by the tissue wall.

The adjustable portion can define a turn in the flow path.

The annular body at the adjustable portion can be formed of an elastic material.

The elastic material can be a cobalt-chromium alloy.

The cardiovascular implant device can be sterilized.

At least a portion of the cardiovascular implant device can be formed of a shape-memory material.

A cardiovascular implant device includes an annular body and one or more anchoring members. The annular body includes a central flow tube extending from an inflow end to an outflow end and a flow path extending through the central flow tube. The one or more anchoring members extend outward from the annular body and are configured to secure the cardiovascular implant device to a tissue wall. The central flow tube is configured to be angled with respect to the tissue wall. The central flow tube is angled to align a flow of blood out of the cardiovascular implant device with a natural flow pattern of blood in a right atrium so that the flow of blood out of the cardiovascular implant device joins with the natural flow pattern of blood in the right atrium.

The cardiovascular implant device of the preceding paragraph can optionally include, additionally and/or alternatively, any one or more of the following features, configurations, and/or additional components:

The outflow end can be configured to be positioned on a right atrial side of an inter-atrial septum when the cardiovascular implant device is positioned in the inter-atrial septum.

The central flow tube can be configured to be angled toward a tricuspid valve plane in the right atrium.

The central flow tube can be configured to direct the flow of blood out of the cardiovascular implant device toward the tricuspid valve plane in the right atrium.

A longitudinal axis through the central flow tube can form an angle of less than ninety degrees (<90°) with a vertical reference plane defined by the tissue wall.

The angle can be between zero and seventy-five degrees (0°-75°).

At least a portion of the central flow tube can be formed by a graft material.

The cardiovascular implant device can be sterilized.

At least a portion of the cardiovascular implant device can be formed of a shape-memory material.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A method of shunting blood between a left atrium and a right atrium of a heart, the method comprising:
positioning a cardiovascular implant device in a tissue wall between the left atrium and the right atrium of the heart, the cardiovascular implant device including a flow directing component;
shunting blood from the left atrium to the right atrium through a flow path of a central flow tube of the cardiovascular implant device;
directing the flow of blood with the flow directing component of the cardiovascular implant device; and
joining the flow of blood through the cardiovascular implant device with a natural flow pattern of blood in the right atrium.

2. The method of claim 1, wherein joining the flow of blood through the cardiovascular implant device with the natural flow pattern of blood in the right atrium further comprises:
joining the flow of blood through the cardiovascular implant device with a vortical flow pattern of blood in the right atrium.

3. The method of claim 2, and further comprising:
enhancing the vortical flow pattern of blood in the right atrium.

4. The method of claim 1, wherein directing the flow of blood with the flow directing component of the cardiovascular implant device further comprises:
directing the flow of blood out of the cardiovascular implant device towards a tricuspid valve plane in the right atrium.

5. The method of claim 1, wherein positioning the cardiovascular implant device in the tissue wall between the left atrium and the right atrium of the heart further comprises:
positioning the flow directing component of the cardiovascular implant device such that a longitudinal axis aligned with the flow of blood out of the cardiovascular implant device forms an angle of about zero to seventy-five degrees (0°-75°) with a vertical reference plane defined by the tissue wall.

6. The method of claim 1, wherein directing the flow of blood with the flow directing component of the cardiovascular implant device further comprises:
directing the flow of blood with a curved portion of the central flow tube adjacent an outflow end of the central flow tube to align the flow of blood out of the cardiovascular implant device with the natural flow pattern of blood in the right atrium.

7. The method of claim 1, wherein directing the flow of blood with the flow directing component of the cardiovascular implant device further comprises:
directing the flow of blood with the central flow tube that is angled with respect to the tissue wall to align the flow of blood out of the cardiovascular implant device with the natural flow pattern of blood in the right atrium.

8. The method of claim 1, wherein positioning the cardiovascular implant device in the tissue wall between the left atrium and the right atrium of the heart further comprises:
positioning the cardiovascular implant device so that the central flow tube is at an angle with respect to the tissue wall.

9. A method of shunting blood between a left atrium and a right atrium of a heart, the method comprising:
positioning a cardiovascular implant device in a tissue wall between the left atrium and the right atrium of the heart, the cardiovascular implant device including a flow directing component that includes a curved portion adjacent an outflow end of a central flow tube;
shunting blood from the left atrium to the right atrium through a flow path of the central flow tube of the cardiovascular implant device;
directing the flow of blood with the flow directing component of the cardiovascular implant device; and
joining the flow of blood through the cardiovascular implant device with a natural flow pattern of blood in the right atrium.

10. The method of claim 9, wherein positioning the cardiovascular implant device in the tissue wall between the left atrium and the right atrium of the heart further comprises:
positioning the curved portion of the cardiovascular implant device to curve towards a tricuspid valve plane in the right atrium.

11. The method of claim 9, wherein joining the flow of blood through the cardiovascular implant device with the natural flow pattern of blood in the right atrium further comprises:
joining the flow of blood through the cardiovascular implant device with a vortical flow pattern of blood in the right atrium.

12. The method of claim 11, and further comprising:
enhancing the vortical flow pattern of blood in the right atrium.

13. The method of claim 9, wherein directing the flow of blood with the flow directing component of the cardiovascular implant device further comprises:
directing the flow of blood out of the cardiovascular implant device towards a tricuspid valve plane in the right atrium.

14. The method of claim 9, wherein positioning the cardiovascular implant device in the tissue wall between the left atrium and the right atrium of the heart further comprises:
positioning the flow directing component of the cardiovascular implant device such that a longitudinal axis aligned with the flow of blood out of the cardiovascular implant device forms an angle of about zero to seventy-five degrees (0°-75°) with a vertical reference plane defined by the tissue wall.

15. A method of shunting blood between a left atrium and a right atrium of a heart, the method comprising:
positioning a cardiovascular implant device in a tissue wall between the left atrium and the right atrium of the heart, the cardiovascular implant device including a flow directing component that includes a central flow tube angled with respect to the tissue wall;
shunting blood from the left atrium to the right atrium through a flow path of the central flow tube of the cardiovascular implant device;
directing the flow of blood with the flow directing component of the cardiovascular implant device; and
joining the flow of blood through the cardiovascular implant device with a natural flow pattern of blood in the right atrium.

16. The method of claim 15, wherein positioning the cardiovascular implant device in the tissue wall between the left atrium and the right atrium of the heart further comprises:
positioning the central flow tube of the cardiovascular implant device to be angled towards a tricuspid valve plane in the right atrium.

17. The method of claim 15, wherein joining the flow of blood through the cardiovascular implant device with the natural flow pattern of blood in the right atrium further comprises:
joining the flow of blood through the cardiovascular implant device with a vortical flow pattern of blood in the right atrium.

18. The method of claim 17, and further comprising:
enhancing the vortical flow pattern of blood in the right atrium.

19. The method of claim 15, wherein directing the flow of blood with the flow directing component of the cardiovascular implant device further comprises:
directing the flow of blood out of the cardiovascular implant device towards a tricuspid valve plane in the right atrium.

20. The method of claim 15, wherein positioning the cardiovascular implant device in the tissue wall between the left atrium and the right atrium of the heart further comprises:
positioning the flow directing component of the cardiovascular implant device such that a longitudinal axis aligned with the flow of blood out of the cardiovascular implant device forms an angle of about zero to seventy-five degrees (0°-75°) with a vertical reference plane defined by the tissue wall.
